SEQUENCE LISTING (27 sequences)

SEQ ID NO: 1 - DNA, 30 bp, Artificial Sequence
  Description of Artificial Sequence Synthetic primer
cuucuauaguuuagguaacuuugtgtttga

SEQ ID NO: 2 - DNA, 36 bp, Artificial Sequence
  Description of Artificial Sequence Synthetic primer
cuucuauaguuuagguaacuuuguguuugannnnnn

SEQ ID NO: 3 - DNA, 45 bp, Artificial Sequence
  Description of Artificial Sequence Synthetic primer
cuucuauaguuuagguaacuuuguguuugatttttttttttttnn

SEQ ID NO: 4 - DNA, 25 bp, Artificial Sequence
  Description of Artificial Sequence Synthetic primer
gguaauacgacucacuauaggcaga

SEQ ID NO: 5 - DNA, 31 bp, Artificial Sequence
  Description of Artificial Sequence Synthetic primer
gguaauacgacucacuauaggcagannnnnn

SEQ ID NO: 6 - DNA, 40 bp, Artificial Sequence
  Description of Artificial Sequence Synthetic primer
gguaauacgacucacuauaggcagatttttttttttttnn

SEQ ID NO: 7 - DNA, 21 bp, Artificial Sequence
  Description of Artificial Sequence Synthetic primer
gacggaugcggucuccagtgt

SEQ ID NO: 8 - DNA, 27 bp, Artificial Sequence
  Description of Artificial Sequence Synthetic primer
gacggaugcggucuccagugunnnnnn

SEQ ID NO: 9 - DNA, 36 bp, Artificial Sequence
  Description of Artificial Sequence Synthetic primer
gacggaugcggucuccagugutttttttttttttnn

SEQ ID NO: 10 - DNA, 22 bp, Artificial Sequence
  Description of Artificial Sequence Synthetic primer
cccuccaaggctccccagtatc

SEQ ID NO: 11 - DNA, 28 bp, Artificial Sequence
  Description of Artificial Sequence Synthetic primer
cccuccaaggctccccaguaucnnnnnn

SEQ ID NO: 12 - DNA, 37 bp, Artificial Sequence
  Description of Artificial Sequence Synthetic primer
cccuccaaggctccccaguauctttttttttttttnn

SEQ ID NO: 13 - DNA, 22 bp, Artificial Sequence
  Description of Artificial Sequence Synthetic primer
agggugagaaaggccggagaca

SEQ ID NO: 14 - DNA, 28 bp, Artificial Sequence
  Description of Artificial Sequence Synthetic primer
agggugagaaaggccggagacannnnnn

SEQ ID NO: 15 - DNA, 37 bp, Artificial Sequence
  Description of Artificial Sequence Synthetic primer
agggugagaaaggccggagacatttttttttttttnn

SEQ ID NO: 16 - DNA, 25 bp, Artificial Sequence
  Description of Artificial Sequence Synthetic primer
acacauacgauuuaggugacactat

SEQ ID NO: 17 - DNA, 31 bp, Artificial Sequence
  Description of Artificial Sequence Synthetic primer
acacauacgauuuaggugacacuaunnnnnn

SEQ ID NO: 18 - DNA, 40 bp, Artificial Sequence
  Description of Artificial Sequence Synthetic primer
acacauacgauuuaggugacacuautttttttttttttnn

SEQ ID NO: 19 - DNA, 25 bp, Artificial Sequence
  Description of Artificial Sequence Synthetic primer
cguuaaguaugaacccagatgtatt

SEQ ID NO: 20 - DNA, 31 bp, Artificial Sequence
  Description of Artificial Sequence Synthetic primer
cguuaaguaugaacccagauguauunnnnnn

SEQ ID NO: 21 - DNA, 40 bp, Artificial Sequence
  Description of Artificial Sequence Synthetic primer
cguuaaguaugaacccagauguauutttttttttttttnn

SEQ ID NO: 22 - DNA, 22 bp, Artificial Sequence
  Description of Artificial Sequence Synthetic primer
gaagacagauggugcagccaca

SEQ ID NO: 23 - DNA, 28 bp, Artificial Sequence
  Description of Artificial Sequence Synthetic primer
gaagacagauggugcagccacannnnnn

SEQ ID NO: 24 - DNA, 37 bp, Artificial Sequence
  Description of Artificial Sequence Synthetic primer
gaagacagauggugcagccacatttttttttttttnn

SEQ ID NO: 25 - DNA, 27 bp, Artificial Sequence
  Description of Artificial Sequence Synthetic primer
cguauucugacgacguacuctcagcct

SEQ ID NO: 26 - DNA, 33 bp, Artificial Sequence
  Description of Artificial Sequence Synthetic primer
cguauucugacgacguacucucagccunnnnnn

SEQ ID NO: 27 - DNA, 42 bp, Artificial Sequence
  Description of Artificial Sequence Synthetic primer
cguauucugacgacguacucucagccutttttttttttttnn

(19) United States
(12) Patent Application Publication
(10) Pub. No.: US 2011/0189679 A1
(43) Pub. Date: Aug. 4, 2011

(54) COMPOSITIONS AND METHODS FOR WHOLE TRANSCRIPTOME ANALYSIS

(75) Inventors: Nurith Kurn, Palo Alto, CA (US); Shenglong Wang, San Ramon, CA (US); Leah R. Turner, Sunnyvale, CA (US); Victor Sementchenko, Santa Clara, CA (US)

(73) Assignee: NuGEN Technologies, Inc., San Carlos, CA (US)

(21) Appl. No.: 12/880,032

(22) Filed: Sep. 10, 2010

Related U.S. Application Data
(60) Provisional application No. 61/241,837, filed on Sep. 11, 2009.

(51) Int. Cl. C12P 19/34; C12Q 1/68

(52) U.S. Cl. 435/6.12; 435/91.51; 435/91.2; 435/6.1

(57) ABSTRACT

The present invention provides methods and compositions, including kits, for the generation of cDNA from mRNA with reduced ribosomal RNA representation.

### CROSS-REFERENCE

This application claims the benefit of U.S. Provisional Application No. 61/241,837, filed Sep. 11, 2009, which application is incorporated herein by reference.

### BACKGROUND OF THE INVENTION

High throughput next generation genome-wide transcriptome analysis methods have been developed in recent years, enabling highly parallel analysis of all transcripts in a given sample, employing a variety of platforms. These platforms include high and low density microarrays and massively parallel sequencing. Analysis of the transcriptome from total RNA samples usually entails reduction or near complete elimination of ribosomal RNA (rRNA) species from the samples. rRNA species represent the majority of RNA in the samples, when compared to all other transcripts. The presence of rRNA may be detrimental to many downstream applications, especially in amplified RNA samples.

Various methods have been applied for rRNA removal. For example Invitrogen has introduced the RiboMinus procedure for removal of rRNA by binding the rRNA to a solid surface by hybridization followed by separation of the solid surface from the samples. Affymetrix Inc. has introduced a procedure for removing rRNA by enzymatic digestions. Others have analyzed only polyA mRNA by purification of the polyA tailed mRNA. These methods, however, are collectively highly inefficient, time consuming and/or expensive and can result in degradation and loss of the mRNA transcripts in the sample. Furthermore, these methods are difficult to perform when analyzing gene expression profiles in minute biological samples.

It is desirable to obtain either amplified or non amplified cDNA from total mRNA in which the amount of rRNA is reduced relative to the original total RNA in the samples, so as to increase the content of non ribosomal RNA transcripts, representing the expressed genes.

### SUMMARY OF THE INVENTION

The present invention provides novel methods and compositions for designing, selecting for, screening, and using specific combinations of reagents that allow for selective cDNA synthesis from an RNA sample, useful for downstream applications such as sequencing and array analyses. Specifically, an important aspect of this invention is the compositions and methods that allow for the reduction or substantial elimination of ribosomal RNA (rRNA) representation in a cDNA sample prepared from RNA.

In one aspect, the invention provides a method for differentially reducing the reverse transcription of ribosomal RNA (rRNA) from an RNA sample. In some embodiments, the method comprises: (a) providing one or more primers of known sequence; (b) combining the one or more primers with a reverse transcriptase (RT); and (c) reverse transcribing the RNA sample, thereby producing reverse transcribed products, wherein the reverse transcription of rRNA is reduced. In some embodiments, the reverse transcription of rRNA is reduced by at least about 10 fold. In some embodiments, the method further comprises step (d) amplifying said reverse transcribed products, thereby producing amplified products. In some embodiments, the method further comprises the step of sequencing the reverse transcribed products. In some embodiments, the reverse transcribed products are amplified prior to sequencing. In some embodiments, the method does not include the step of purification of one or more RNA sequences from the sample.

In one aspect, the invention provides a method of identifying a primer sequence that differentially reduces the reverse transcription of ribosomal RNA (rRNA) from an RNA sample. In some embodiments, the method comprises (a) providing one or more primers of known sequence; (b) combining the one or more primers with a reverse transcriptase (RT) to reverse transcribe the RNA sample, thereby producing reverse transcribed products; (c) optionally generating double-stranded cDNA products from said reverse transcribed products; (d) optionally amplifying said double-stranded cDNA products; and (e) analyzing the reverse transcribed products to determine if the reverse transcription of rRNA is reduced by the primer sequence.

In one aspect, the invention provides a method of identifying a reverse transcriptase enzyme (RT) that differentially reduces the reverse transcription of rRNA from an RNA sample. In some embodiments, the method comprises (a) providing one or more primers of known sequence; (b) using the primer with an RT to reverse transcribe the RNA sample, thereby producing reverse transcribed products; and (c) optionally generating double-stranded cDNA products from said reverse transcribed products; (d) optionally amplifying said double-stranded cDNA products; and (e) analyzing the reverse transcribed products to determine if the reverse transcription of rRNA is reduced by the RT.

In one aspect, the invention provides for a method for whole transcriptome sequencing comprising providing a RNA sample, reverse transcribing the sample, amplifying the RNA sample using one or more primers to produce amplified products, and performing sequencing on the products. In some embodiments, the method does not include a) pre-treatment of the RNA sample, b) prior selection of the RNA sample, c) selection of sequences from the RNA sample comprising a polyA sequence, d) prior removal of ribosomes from the RNA sample, or d) substantial loss of the RNA in the sample. In some embodiments, the method does not include the step of purification of one or more RNA sequences from the sample.

In one aspect, the invention provides for a collection of chimeric primers, each comprising RNA and DNA, and a RT enzyme, wherein the RT is used in combination with the chimeric primers to reverse transcribe a whole transcriptome. In such an embodiment, no more than 5%, 10%, 15%, 20%, 25%, 30%, 40%, 50%, 60%, 70%, 75%, or 80% of the resulting amplicons are rRNA sequences.

In some embodiments, the one or more primers are tailed primers comprising a 3′ portion hybridizable to one or more target RNAs in the RNA sample and a 5′ portion that is not hybridizable to the one or more target RNAs in the RNA sample. In some embodiments, the one or more primers are chimeric primers comprising a DNA portion and an RNA portion. In some embodiments, the chimeric primers comprise a 3′-DNA portion hybridizable to one or more target RNAs in the RNA sample and a 5′-RNA portion that is not hybridizable to the one or more target RNAs in the RNA sample. The hybridizable 3′ portion of the primers can comprise a randomly generated sequence. The hybridizable 3′ portion of the primer can comprise 5-15 nucleotides, for example 5, 6, 7, 8, 9, 10, 11, 12, 13, 14, or 15 nucleotides. The non-hybridizable 5′ portion of the primers can comprise a promoter-specific sequence. In some embodiments, the primer is one of a plurality of primers of known sequence, each primer comprising a 3′ portion hybridizable to the sample RNA and a 5′ portion that is not hybridizable to the sample RNA, wherein each of the non-hybridizable 5′ portions of the primers comprise the same sequence.

In some embodiments, the RT is selected from the group consisting of: a Moloney murine leukemia virus RT, a human immunodeficiency virus RT, a rous sarcoma virus RT, an avian myeloblastosis virus RT, a rous associated virus RT, a myeloblastosis associated virus RT, an avian sarcoma-leukosis virus RT, an RT lacking RNase H activity, modified RTs derived therefrom, and combinations thereof.

In some embodiments, the reverse transcription of rRNA is reduced by at least about 20%, at least about 30%, at least about 40%, or at least about 50%. In some embodiments, the reverse transcription of rRNA is reduced by at least about 2-fold. In some embodiments, the sequence that reduces the reverse transcription of ribosomal RNA is identified based on varying the non-hybridizable 5′ of the one or more primers. In some embodiments, the primer sequence that reduces the reverse transcription of ribosomal RNA is identified based on varying the RT used in step (c).

In some embodiments, the RNA sample comprises mRNA and rRNA. In some embodiments, the RNA is selected from the group consisting of total RNA, mitochondrial RNA, chloroplast RNA, DNA-RNA hybrids, viral RNA, cell free RNA, and mixtures thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts the effect of first strand tailed primers on non amplified cDNA generation by RTa.

FIG. 2 depicts the effect of first strand tailed primers on rRNA content in non-amplified cDNA generation by RTb.

FIG. 3 depicts that mitochondrial rRNA and transcripts are affected to a lesser extent by choice of first strand tailed primer: RTa.

FIG. 4 depicts that mitochondrial rRNA and transcripts are affected to a lesser extent by choice of first strand tailed primer: RTb.

FIG. 5 depicts non amplified cDNA, where delta Ct represents Ct for RTa minus Ct for RTb.

FIG. 6 depicts non amplified cDNA, where delta CT represents Ct for RTa minus Ct for RTb.

FIG. 7 depicts non amplified cDNA generated by RTa: rRNA avg. Ct.

FIG. 8 depicts non-amplified cDNA generated by RTb: rRNA avg. Ct.

FIG. 9 depicts non-amplified cDNA generated by RTa: mt rRNA avg. Ct.

FIG. 10 depicts non-amplified cDNA generated by RTb: mt rRNA avg. Ct.

FIG. 11 depicts quantification of rRNA in amplified cDNA.

FIG. 12 depicts quantification of various mRNAs in amplified cDNA (average rRNA Ct is given as a reference).

FIG. 13 depicts quantification of mitochondrial RNA in amplified cDNA.

FIG. 14 depicts quantification of mitochondrial transcripts in amplified cDNA.

FIG. 15 depicts a comparison of cDNA prepared with RTa or RTb and SPIA-amplified using WGA-Ovation.

FIG. 16 depicts a comparison of cDNA generated by RTa or RTb and SPIA-amplified using Pico-Ovation SPIA.

FIG. 17 depicts a comparison of SPIA amplification reagents: cDNA generated with RTa and amplified with Pico- or WGA-Ovation SPIA.

FIG. 18 depicts a comparison of SPIA amplification reagents: cDNA generated with RTb and amplified with Pico- or WGA-Ovation SPIA.

FIG. 19 depicts differences in rRNA representation by comparison of depth of coverage.

FIG. 20 depicts differences in rRNA representation by comparison of depth of coverage.

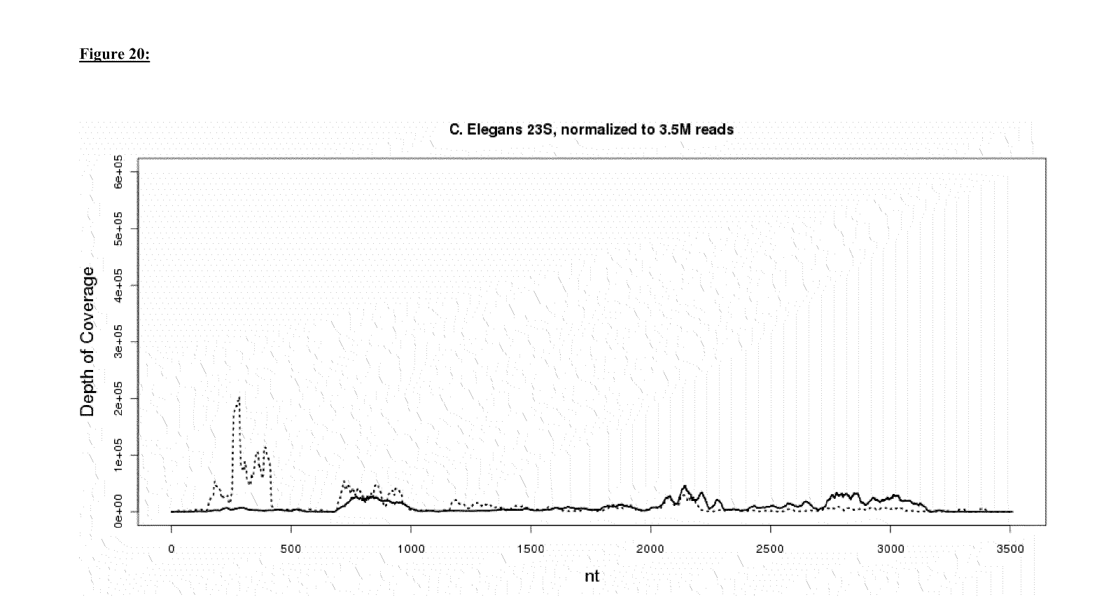

### DETAILED DESCRIPTION OF THE INVENTION

### General

In one embodiment, the present invention provides methods and compositions for selectively amplifying a target population of nucleic acid molecules (e.g. all mRNA molecules expressed in a cell except for the most highly expressed mRNA species). In another embodiment, the present invention provides methods and compositions for screening for and/or selecting suitable primers and reverse transcriptases (RTs) which are capable of selectively reducing the cDNA generation of rRNA, while maintaining the representation of non-rRNA transcripts. The methods further enable generation of cDNA which can be further amplified by a variety of amplification methods, for example, the single primer isothermal linear amplification method (SPIA).

### Primers

A “primer,” as used herein, refers to a nucleotide sequence, generally with a free 3′-OH group, that hybridizes with a template sequence (such as one or more target RNAs, or a primer extension product) and is capable of promoting polymerization of a polynucleotide complementary to the template. A “primer” can be, for example, an oligonucleotide. It can also be, for example, a sequence of the template (such as a primer extension product or a fragment of the template created following RNase cleavage of a template-DNA complex) that is hybridized to a sequence in the template itself (for example, as a hairpin loop), and that is capable of promoting nucleotide polymerization. Thus, a primer can be an exogenous (e.g., added) primer or an endogenous (e.g., template fragment) primer. A primer may contain a non-hybridizing sequence that constitutes a tail on the primer. A primer may still be hybridizing even though its sequences are not completely complementary to the target.

The primers of the invention are usually oligonucleotide primers. A primer is generally an oligonucleotide that is employed in an extension by a polymerase along a polynucleotide template such as in, for example, PCR or SPIA. The oligonucleotide primer is often a synthetic polynucleotide that is single stranded, containing a sequence at its 3′-end that is capable of hybridizing with a sequence of the target polynucleotide. Normally, the 3′ region of the primer that hybridizes with the target nucleic acid has at least 80%, preferably 90%, more preferably 95%, most preferably 100%, complementarity to a sequence or primer binding site. “Complementary”, as used herein, refers to complementarity to all or only to a portion of a sequence. The number of nucleotides in the hybridizable sequence of a specific oligonucleotide primer should be such that stringency conditions used to hybridize the oligonucleotide primer will prevent excessive random non-specific hybridization. Usually, the number of nucleotides in the hybridizing portion of the oligonucleotide primer will be at least as great as the defined sequence on the target polynucleotide that the oligonucleotide primer hybridizes to, namely, at least 5, at least 6, at least 7, at least 8, at least 9, at least 10, at least 11, at least 12, at least 13, at least 14, at least 15, at least about 20, and generally from about 6 to about 10 or 6 to about 12 of 12 to about 200 nucleotides, usually about 20 to about 50 nucleotides. In general, the target polynucleotide is larger than the oligonucleotide primer or primers as described previously. In some cases, the hybridizable sequence of an oligonucleotide primer is a random sequence. Oligonucleotide primers comprising random sequences may be referred to as random primers, as described herein. In other cases, an oligonucleotide primer such as a first primer or a second primer comprises a set of primers such as for example a set of first primers or a set of second primers. In some cases, the set of first or second primers may comprise a mixture of primers designed to hybridize to a plurality (e.g. 2, 3, 4, about 6, 8, 10, 20, 40, 80, 100, 125, 150, 200, 250, 300, 400, 500, 600, 800, 1000, 1500, 2000, 2500, 3000, 4000, 5000, 6000, 7000, 8000, 10,000, 20,000, 25,000 or more) of target sequences. In some cases, the plurality of target sequences may comprise a group of related sequences, random sequences, a whole transcriptome or fraction (e.g. substantial fraction) thereof, or any group of sequences such as mRNA.

Tailed primers are employed in certain embodiments of the invention. In general, a tailed primer comprises a 3′ portion that is hybridizable to one or more target polynucleotides, such as one or more target RNAs in an RNA sample, and a 5′ portion that is not hybridizable to the one or more target polynucleotides. In general, the non-hybridizable 5′ portion does not hybridize to the one or more target polynucleotides under conditions in which the hybridizable 3′ portion of the tailed primer hybridizes to the one or more target polynucleotides. In some embodiments, the non-hybridizable 5′ portion comprises a promoter-specific sequence. Generally, a promoter-specific sequence comprises a single-stranded DNA sequence region which, in double-stranded form is capable of mediating RNA transcription. Examples of promoter-specific sequences are known in the art, and include, without limitation, T7, T3, or SP6 RNA polymerase promoter sequences. When the tailed primer is extended with a DNA polymerase, a primer extension product with a 5′ portion comprising a defined sequence can be created. This primer extension product can then have a second primer anneal to it, which can be extended with a DNA polymerase to create a double stranded product comprising a defined sequence at one end. In some embodiments, where the non-hybridizable 5′ portion of one or more tailed primers comprises a promoter-specific sequence, creation of a double-stranded product comprising a defined sequence at one end generates a double-stranded promoter sequence that is capable of mediating RNA transcription. In some embodiments, a double-stranded promoter sequence is generated by hybridizing to the promoter-specific sequence an oligonucleotide comprising a sequence complementary to the promoter-specific sequence. In some embodiments, formation of a double-stranded promoter is followed by the generation of single-stranded RNA by RNA transcription of sequence downstream of the double-stranded promoter, generally in a reaction mixture comprising all necessary components, including but not limited to ribonucleoside triphosphates (rNTPs) and a DNA-dependent RNA polymerase. Tailed primers can comprise DNA, RNA, or both DNA and RNA. In some embodiments, the tailed primer consists of DNA.

Composite primers are employed in certain embodiments of the invention. Composite primers are primers that are composed of RNA and DNA portions. In some aspects, the composite primer is a tailed composite primer comprising, for example, a 3′-DNA portion and a 5′-RNA portion. In the tailed composite primer, a 3′-portion, all or a portion of which comprises DNA, is complementary to a polynucleotide; and a 5′-portion, all or a portion of which comprises RNA, is not complementary to the polynucleotide and does not hybridize to the polynucleotide under conditions in which the 3′-portion of the tailed composite primer hybridizes to the polynucleotide target. When the tailed composite primer is extended with a DNA polymerase, a primer extension product with a 5′-RNA portion comprising a defined sequence can be created. This primer extension product can then have a second primer anneal to it, which can be extended with a DNA polymerase to create a double stranded product with an RNA/DNA heteroduplex comprising a defined sequence at one end. The RNA portion can be selectively cleaved from the partial heteroduplex to create a double-stranded DNA with a 3′-single-stranded overhang which can be useful for various aspects of the present invention including allowing for isothermal amplification using a composite amplification primer.

In other aspects, the composite primer is an amplification composite primer (interchangeably called composite amplification primer). In the amplification composite primer, both the RNA and the DNA portions are generally complementary and hybridize to a sequence in the polynucleotide to be copied or amplified. In some embodiments, a 3′-portion of the amplification composite primer is DNA and a 5′-portion of the composite amplification primer is RNA. The composite amplification primer is designed such that the primer is extended from the 3′-DNA portion to create a primer extension product. The 5′-RNA portion of this primer extension product, in a RNA/DNA heteroduplex is susceptible to cleavage by RNase H, thus freeing a portion of the polynucleotide to the hybridization of an additional composite amplification primer. The extension of the amplification composite primer by a DNA polymerase with strand displacement activity releases the primer extension product from the original primer and creates another copy of the sequence of the polynucleotide. Repeated rounds of primer hybridization, primer extension with strand displacement DNA synthesis, and RNA cleavage create multiple copies of the sequence of the polynucleotide. Composite primers are described in more detail below.

A “random primer,” as used herein, is a primer that generally comprises a sequence that is designed not necessarily based on a particular or specific sequence in a sample, but rather is based on a statistical expectation (or an empirical observation) that the sequence of the random primer is hybridizable (under a given set of conditions) to one or more sequences in the sample. A random primer will generally be an oligonucleotide or a population of oligonucleotides comprising a random sequence(s) in which the nucleotides at a given position on the oligonucleotide can be any of the four nucleotides, or any of a selected group of the four nucleotides (for example only three of the four nucleotides, or only two of the four nucleotides). In some cases all of the positions of the oligonucleotide or population of oligonucleotides can be any of two or more nucleotides. In other cases, only a portion of the oligonucleotide, for instance a particular region, will comprise positions which can be any of two or more bases. In some cases, the portion of the oligonucleotide which comprises positions which can be any of two or more bases is about 5, 6, 7, 8, 9, 10, 11, 12, 13, 14, or about 15-20 nucleotides in length. In some cases, a random primer may comprise a tailed primer having a 3′-region that comprises a random sequence and a 5′-region that is a non-hybridizing sequence that comprises a specific, non-random sequence. The 3′-region may also comprise a random sequence in combination with a region that comprises poly-T sequences. The sequence of a random primer (or its complement) may or may not be naturally-occurring, or may or may not be present in a pool of sequences in a sample of interest. The amplification of a plurality of RNA species in a single reaction mixture can employ, but not necessarily employ, a multiplicity, preferably a large multiplicity, of random primers. As is well understood in the art, a “random primer” can also refer to a primer that is a member of a population of primers (a plurality of random primers) which collectively are designed to hybridize to a desired and/or a significant number of target sequences. A random primer may hybridize at a plurality of sites on a nucleic acid sequence. The use of random primers provides a method for generating primer extension products complementary to a target polynucleotide which does not require prior knowledge of the exact sequence of the target. In some embodiments one portion of a primer is random, and another portion of the primer comprises a defined sequence. For example, in some embodiments, a 3′-portion of the primer will comprise a random sequence, while the 5′-portion of the primer comprises a defined sequence. In some embodiments a 3′-random portion of the primer will comprise DNA, and a 5′-defined portion of the primer will comprise RNA, in other embodiments, both the 3′ and 5′-portions will comprise DNA. In some embodiments, the 5′-portion will contain a defined sequence and the 3′-portion will comprise a poly-dT sequence that is hybridizable to a multiplicity of RNAs in a sample (such as all mRNA). In some embodiments, a “random primer,” or primer comprising a randomly generated sequence, comprises a collection of primers comprising one or more nucleotides selected at random from two or more different nucleotides, such that all possible sequence combinations of the nucleotides selected at random may be represented in the collection. In some embodiments, generation of one or more random primers does not include a step of excluding or selecting certain sequences or nucleotide combinations from the possible sequence combinations in the random portion of the one or more random primers.

In one embodiment, the preferred primers of the invention are tailed primers. In this embodiment, the 5′-tail can comprise RNA and is non hybridizable to the RNA in the sample. The 3′-end of the first primer(s) can be hybridizable to the RNA in the sample, comprise DNA and comprise a random sequence, enabling hybridization across the whole transcriptome. The first primer may also comprise a mixture of primers. The mixture of first primers may also include a first primer comprising a 3′-DNA sequence hybridizable to the 3′-poly A tail of mRNA, in addition to the first primers comprising a random sequence at the 3′-ends.

### RNA Dependent DNA Polymerases

RNA-dependent DNA polymerases for use in the methods and compositions of the invention are capable of effecting extension of a primer according to the methods of the invention. Accordingly, a preferred RNA-dependent DNA polymerase is one that is capable of extending a nucleic acid primer along a nucleic acid template that is comprised at least predominantly of ribonucleotides. Suitable RNA-dependent DNA polymerases for use in the methods and compositions of the invention include reverse transcriptases (RTs). RTs are well known in the art. Examples of RTs include, but are not limited to, Moloney murine leukemia virus (M-MLV) reverse transcriptase, human immunodeficiency virus (HIV) reverse transcriptase, rous sarcoma virus (RSV) reverse transcriptase, avian myeloblastosis virus (AMV) reverse transcriptase, rous associated virus (RAV) reverse transcriptase, and myeloblastosis associated virus (MAV) reverse transcriptase or other avian sarcoma-leukosis virus (ASLV) reverse transcriptases, and modified RTs derived therefrom. See e.g. U.S. Pat. No. 7,056,716. Many reverse transcriptases, such as those from avian myeoloblastosis virus (AMV-RT), and Moloney murine leukemia virus (MMLV-RT) comprise more than one activity (for example, polymerase activity and ribonuclease activity) and can function in the formation of the double stranded cDNA molecules. However, in some instances, it is preferable to employ a RT which lacks or has substantially reduced RNase H activity. RTs devoid of RNase H activity are known in the art, including those comprising a mutation of the wild type reverse transcriptase where the mutation eliminates the RNase H activity. Examples of RTs having reduced RNase H activity are described in US20100203597. In these cases, the addition of an RNase H from other sources, such as that isolated from E. coli, can be employed for the formation of the double stranded cDNA. Combinations of RTs are also contemplated, including combinations of different non-mutant RTs, combinations of different mutant RTs, and combinations of one or more non-mutant RT with one or more mutant RT.

### Methods of Amplification

Methods of nucleic acid amplification are well known in the art.

In some aspects of the invention, the amplification method that is used to amplify the marked DNA is a single primer isothermal amplification (SPIA) using a complex comprising a RNA/DNA partial heteroduplex as a template. In this method a complex comprising a RNA/DNA partial heteroduplex is a substrate for further amplification as follows: an enzyme (such as RNase H), which cleaves RNA sequence from an RNA/DNA heteroduplex, cleaves the RNA sequence (A) from the partial heteroduplex, leaving a partially double stranded polynucleotide complex comprising an exposed 3′ single-stranded DNA sequence. The 3′ single-stranded sequence (formed by cleavage of RNA in the complex comprising a RNA/DNA partial heteroduplex) is generally the complement of a composite amplification primer, and thus forms a specific binding site for the composite amplification primer. This composite amplification primer is the third primer used in this invention and also comprises a unique 5′-sequence. Extension of a bound composite amplification primer by a DNA-dependent DNA polymerase with strand displacement activity produces a primer extension product, which displaces the previously bound cleaved primer extension product, whereby single stranded DNA product accumulates. The single stranded DNA product is a copy of the complement of the target RNA (or “antisense” DNA). The cycle repeats with the removal of the unique sequence on the 5′-tail of the primer extension product, exposing the 3′-end of the second primer extension product for another cycle of amplification. This linear amplification is referred to as “SPIA” (for Single Primer Linear Isothermal Amplification), and is described in Kurn et al., U.S. Pat. Nos. 6,251,639 and 6,692,918.

Amplification using a complex comprising a RNA/DNA partial heteroduplex as a template for further amplification by SPIA generally occurs under conditions permitting composite primer hybridization, primer extension by a DNA polymerase with strand displacement activity, cleavage of RNA from a RNA/DNA heteroduplex and strand displacement. In so far as the composite amplification primer hybridizes to the 3′-single-stranded portion (of the partially double stranded polynucleotide which is formed by cleaving RNA in the complex comprising a RNA/DNA partial heteroduplex) comprising, generally, the complement of at least a portion of the composite amplification primer sequence, composite primer hybridization may be under conditions permitting specific hybridization.

In some embodiments, the methods of the invention result in amplification of a multiplicity, a large multiplicity, or a very large multiplicity of target RNA. In some embodiments, essentially all of the RNA present in the initial sample (e.g., all of the mRNA) substantially free of rRNA is amplified. In other embodiments, at least 1, at least 5, at least 10, at least 20, at least 50, at least 100, at least 200, at least 300, up to at least 10,000 or more distinct sequences (such as other sub-segments of a RNA) are amplified (also substantially free of rRNA), as assessed, e.g., by analysis of marker sequences known to be present in the template sample under analysis, using methods known in the art. Target RNA sequences that are amplified may be present on the same RNA strand or on different RNA strands. It will be understood by those of skill in the art that the global amplification methods of the invention are suitable for amplification of any pool or subset of RNA.

In some embodiments, the methods of the invention are used to globally amplify a single stranded RNA target or the double stranded DNA that is initially produced from the RNA target using methods described herein. In these cases, the amplification product will generally be a copy of either the target RNA (sense copy) or of the complement to the target RNA (antisense copy). Whether the sense or antisense copy is produced will depend on the method, as will be understood by one of ordinary skill in the art. In some embodiments, the amplification product of different senses can be annealed to form a double-stranded (or partially double-stranded) complex. In other embodiments, they can be prevented from annealing (or subsequently denatured) to produce a mixture of single stranded amplification products. The amplified products may be of differing lengths.

As illustrated in these embodiments, all steps are isothermal (in the sense that thermal cycling is not required), although the temperatures for each of the steps may or may not be the same. It is understood that various other embodiments may be practiced, given the general description provided above. For example, as described and exemplified herein, certain steps may be performed as temperature is changed (e.g., raised, or lowered).

For simplicity, the isothermal amplification methods of the invention are described as two distinct steps or phases, above. It is understood that the two phases may occur simultaneously in some embodiments (for example, if the enzyme that cleaves RNA from RNA/DNA heteroduplex is included in the first reaction mixture).

Although generally only one composite amplification primer is described above, it is further understood that the amplification methods may be performed in the presence of two or more different first and/or second composite primers that randomly prime template polynucleotide. In addition, the amplification polynucleotide products of two or more separate amplification reactions conducted using two or more different first and/or second composite primers that randomly prime template polynucleotide can be combined.

Methods of Screening for Reagents for Selective cDNA Synthesis

The compositions and methods provided herein are useful for screening for a combination of reagents that will yield a desired result.

By way of an example, an optimal RT can be screened for to achieve a reduction in the reverse transcription and/or subsequent amplification of rRNA by at least about 5%, at least about 10%, at least about 20%, at least about 25%, at least about 30%, at least about 40%, at least about 50%, at least about 60%, at least about 70%, at least about 75%, at least about 80%, at least about 85%, at least about 90%, at least about 95%, at least about 99%, or more. By way of further example, an optimal RT can be screened for to achieve a reduction in the reverse transcription and/or subsequent amplification of rRNA by at least about 2-fold, 3-fold, 4-fold, 5-fold, 6-fold, 7-fold, 8-fold, 10-fold, 100-fold, 200-fold, 300-fold, 400-fold, 500-fold, 600-fold, 700-fold, 800-fold, 900-fold, 1000-fold, 10.000-fold, or more.

By way of another example, an optimal reverse transcription primer sequence, such as a tailed primer, a chimeric primer comprising RNA and DNA sequences, or a tailed chimeric primer, can be screened for to achieve a reduction in the reverse transcription and/or subsequent amplification of rRNA by at least about 5%, at least about 10%, at least about 20%, at least about 25%, at least about 30%, at least about 40%, at least about 50%, at least about 60%, at least about 70%, at least about 75%, at least about 80%, at least about 85%, at least about 90%, at least about 95%, at least about 99%, or more. By way of further example, an optimal reverse transcription primer sequence can be screened for to achieve a reduction in the reverse transcription and/or subsequent amplification of rRNA by at least about 2-fold, 3-fold, 4-fold, 5-fold, 6-fold, 7-fold, 8-fold, 10-fold, 100-fold, 200-fold, 300-fold, 400-fold, 500-fold, 600-fold, 700-fold, 800-fold, 900-fold, 1000-fold, 10.000-fold, or more.

By way of yet another example, an optimal combination of reverse transcription primer and RT can be screened for to achieve a reduction in the reverse transcription and/or subsequent amplification of rRNA by at least about 5%, at least about 10%, at least about 20%, at least about 25%, at least about 30%, at least about 40%, at least about 50%, at least about 60%, at least about 70%, at least about 75%, at least about 80%, at least about 85%, at least about 90%, at least about 95%, or at least about 99%. In some embodiments, reverse transcription and/or subsequent amplification of rRNA is reduced by at least about 2-fold, 3-fold, 4-fold, 5-fold, 6-fold, 7-fold, 8-fold, 10-fold, 100-fold, 200-fold, 300-fold, 400-fold, 500-fold, 600-fold, 700-fold, 800-fold, 900-fold, 1000-fold, 10.000-fold, or more.

In some embodiments, a reduction in the reverse transcription and/or subsequent amplification of rRNA is with respect to an amount, fraction, or representation of rRNA in the starting sample prior to reverse transcription and/or amplification. In some embodiments, a reduction in the reverse transcription and/or subsequent amplification of rRNA is with respect to an amount, fraction, or representation of rRNA in a control sample, wherein RNA is reverse transcribed or subsequently amplified using one or more non-selective agents, such as a pool of random hexamer, heptamter, octomer, 9-mer, or N-mer primers (e.g. with each position of every primer in the pool selected at random, preferably representing all combinations of sequences of length N, the length of the random primer).

In some embodiments, the method does not include a step for the purification of one or more RNA sequences from the sample, such as the removal of a non-target RNA, or the separation of target RNA. Non-target RNA can include rRNA.

### Downstream Applications for Whole Transcriptome Analysis

An important aspect of the invention is that the methods and compositions disclosed herein can be efficiently and cost-effectively utilized for downstream analyses, such as next-generation sequencing or hybridization platforms, with minimal loss of biological material of interest. In one embodiment, a purpose of the embodiments herein is to reduce rRNA content from total RNA, i.e. from the whole transcriptome.

The methods of the invention are useful, for example, for efficient sequencing of a polynucleotide sequence of interest. Specifically the methods of the invention are useful for massively parallel sequencing of a product substantially free of rRNA sequences.

In one embodiment, the invention provides for a method for whole transcriptome sequencing comprising providing a RNA sample, providing one or more primers of known sequence, combining the one or more primers with a reverse transcriptase, reverse transcribing the sample, amplifying the RNA sample using one or more primers to produce amplified products, and performing sequencing on the products. In this embodiment, the method preferably does not include one or more of: (a) pre-treatment of RNA sample, (b) prior selection of the RNA sample, (c) selection of sequences from the RNA sample comprising a polyA sequence, (d) prior removal of ribosomes from the RNA sample, (e) substantial loss of the RNA in the sample, (f) purification of target RNA from the sample, and (g) purification of non-target RNA from the sample. In some embodiments, sequencing is performed on single- or double-stranded cDNA generated by the reverse transcription of an RNA sample without amplifying the RNA sample. In some embodiments, the starting amount of RNA is 0.01 ng to 100 mg. The primers used for reverse transcription and/or amplification can be tailed primers, chimeric primers, or tailed and chimeric primers.

In one embodiment, a collection of tailed primers, and a RT enzyme is provided, wherein the RT is used in combination with the tailed primers to reverse transcribe a whole transcriptome. In one embodiment, a collection of chimeric primers, each comprising RNA and DNA, and a RT enzyme is provided, wherein the RT is used in combination with the chimeric primers to reverse transcribe a whole transcriptome. In some embodiment, no more than 5%, 10%, 15%, 20%, 25%, 30%, 40%, 50%, 60%, 70%, 75%, 80% of the resulting products are rRNA sequences.

Known methods for sequencing include, for example, those described in: Sanger, F. et al., Proc. Natl. Acad. Sci. U.S.A. 75, 5463-5467 (1977); Maxam, A. M. & Gilbert, W. Proc Natl Acad Sci USA 74, 560-564 (1977); Ronaghi, M. et al., Science 281, 363, 365 (1998); Lysov, l. et al., Dokl Akad Nauk SSSR 303, 1508-1511 (1988); Bains W. & Smith G. C. J. Theor Biol 135, 303-307 (1988); Drnanac, R. et al., Genomics 4, 114-128 (1989); Khrapko, K. R. et al., FEBS Lett 256.118-122 (1989); Pevzner P. A. J Biomol Struct Dyn 7, 63-73 (1989); and Southern, E. M. et al., Genomics 13, 1008-1017 (1992). Pyrophosphate-based sequencing reaction as described, e.g., in U.S. Pat. Nos. 6,274,320,6258, 568 and 6,210,891), may also be used. In some cases, the methods above require that the nucleic acid attached to the solid surface be single stranded. In such cases, the unbound strand may be melted away using any number of commonly known methods such as addition of NaOH, application of low ionic (e.g., salt) strength solution, enzymatic degradation or displacement of the second strand, or heat processing. Where the solid surface comprises a plurality of beads, following this strand removal step, the beads can be pelleted and the supernatant discarded. The beads can then be resuspended in a buffer, and a sequencing primer or other non-amplification primer can be added. The primer is annealed to the single stranded amplification product. This can be accomplished by using an appropriate annealing buffer and temperature conditions, e.g., as according to standard procedures in the art.

The methods of the invention are useful, for example, for sequencing of an RNA sequence of interest. The sequencing process can be carried out by processing and amplifying a target RNA containing the sequence of interest by any of the methods described herein. Addition of nucleotides during primer extension can be analyzed using methods known in the art, for example, incorporation of a terminator nucleotide, sequencing by synthesis (e.g. pyrosequencing), or sequencing by ligation.

In embodiments wherein the end product is in the form of DNA primer extension products, in addition to the nucleotides, such as natural deoxyribonucleotide triphosphates (dNTPs), that are used in the amplification methods, appropriate nucleotide triphosphate analogs, which may be labeled or unlabeled, that upon incorporation into a primer extension product effect termination of primer extension, may be added to the reaction mixture. Preferably, the dNTP analogs are added after a sufficient amount of reaction time has elapsed since the initiation of the amplification reaction such that a desired amount of second primer extension product or fragment extension product has been generated. Said amount of the time can be determined empirically by one skilled in the art.

Suitable dNTP analogs include those commonly used in other sequencing methods and are well known in the art. Examples of dNTP analogs include dideoxyribonucleotides. Examples of rNTP analogs (such as RNA polymerase terminators) include 3′-dNTP. Sasaki et al., Biochemistry (1998) 95:3455-3460. These analogs may be labeled, for example, with fluorochromes or radioisotopes. The labels may also be labels which are suitable for mass spectroscopy. The label may also be a small molecule which is a member of a specific binding pair, and can be detected following binding of the other member of the specific binding pair, such as biotin and streptavidin, respectively, with the last member of the binding pair conjugated to an enzyme that catalyzes the generation of a detectable signal that could be detected by methods such as colorimetry, fluorometry or chemiluminescence. All of the above examples are well known in the art. These are incorporated into the primer extension product or RNA transcripts by the polymerase and serve to stop further extension along a template sequence. The resulting truncated polymerization products are labeled. The accumulated truncated products vary in length, according to the site of incorporation of each of the analogs, which represent the various sequence locations of a complementary nucleotide on the template sequence.

Analysis of the reaction products for elucidation of sequence information can be carried out using any of various methods known in the art. Such methods include gel electrophoresis and detection of the labeled bands using appropriate scanner, sequencing gel electrophoresis and detection of the radiolabeled band directly by phosphorescence, capillary electrophoresis adapted with a detector specific for the labels used in the reaction, and the like. The label can also be a ligand for a binding protein which is used for detection of the label in combination with an enzyme conjugated to the binding protein, such as biotin-labeled chain terminator and streptavidin conjugated to an enzyme. The label is detected by the enzymatic activity of the enzyme, which generates a detectable signal. As with other sequencing methods known in the art, the sequencing reactions for the various nucleotide types (A, C, G, T or U) are carried out either in a single reaction vessel, or in separate reaction vessels (each representing one of the various nucleotide types). The choice of method to be used is dependent on practical considerations readily apparent to one skilled in the art, such as the nucleotide tri phosphate analogs and/or label used. Thus, for example, when each of the analogs is differentially labeled, the sequencing reaction can be carried out in a single vessel. The considerations for choice of reagent and reaction conditions for optimal performance of sequencing analysis according to the methods of the invention are similar to those for other previously described sequencing methods. The reagent and reaction conditions should be as described above for the nucleic acid amplification methods of the invention.

Other examples of template dependent sequencing methods include sequence by synthesis processes, where individual nucleotides are identified iteratively, as they are added to the growing primer extension product.

Pyrosequencing is an example of a sequence by synthesis process that identifies the incorporation of a nucleotide by assaying the resulting synthesis mixture for the presence of by-products of the sequencing reaction, namely pyrophosphate. In particular, a primer/template/polymerase complex is contacted with a single type of nucleotide. If that nucleotide is incorporated, the polymerization reaction cleaves the nucleoside triphosphate between the α and β phosphates of the triphosphate chain, releasing pyrophosphate. The presence of released pyrophosphate is then identified using a chemiluminescent enzyme reporter system that converts the pyrophosphate, with AMP, into ATP, then measures ATP using a luciferase enzyme to produce measurable light signals. Where light is detected, the base is incorporated, where no light is detected, the base is not incorporated. Following appropriate washing steps, the various bases are cyclically contacted with the complex to sequentially identify subsequent bases in the template sequence. See, e.g., U.S. Pat. No. 6,210,891, incorporated herein by reference in its entirety for all purposes).

In related processes, the primer/template/polymerase complex is immobilized upon a substrate and the complex is contacted with labeled nucleotides. The immobilization of the complex may be through the primer sequence, the template sequence and/or the polymerase enzyme, and may be covalent or noncovalent. In general, preferred aspects, particularly in accordance with the invention provide for immobilization of the complex via a linkage between the polymerase or the primer and the substrate surface. A variety of types of linkages are useful for this attachment, including, e.g., provision of biotinylated surface components, using e.g., biotin-PEG-silane linkage chemistries, followed by biotinylation of the molecule to be immobilized, and subsequent linkage through, e.g., a streptavidin bridge. Other synthetic coupling chemistries, as well as non-specific protein adsorption can also be employed for immobilization. In alternate configurations, the nucleotides are provided with and without removable terminator groups. Upon incorporation, the label is coupled with the complex and is thus detectable. In the case of terminator bearing nucleotides, all four different nucleotides, bearing individually identifiable labels, are contacted with the complex. Incorporation of the labeled nucleotide arrests extension, by virtue of the presence of the terminator, and adds the label to the complex. The label and terminator are then removed from the incorporated nucleotide, and following appropriate washing steps, the process is repeated. In the case of non-terminated nucleotides, a single type of labeled nucleotide is added to the complex to determine whether it will be incorporated, as with pyrosequencing. Following removal of the label group on the nucleotide and appropriate washing steps, the various different nucleotides are cycled through the reaction mixture in the same process. See, e.g., U.S. Pat. No. 6,833,246, incorporated herein by reference in its entirety for all purposes). For example, the Illumina Genome Analyzer System is based on technology described in WO 98/44151, hereby incorporated by reference, wherein DNA molecules are bound to a sequencing platform (flow cell) via an anchor probe binding site (otherwise referred to as a flow cell binding site) and amplified in situ on a glass slide. The DNA molecules are then annealed to a sequencing primer and sequenced in parallel base-by-base using a reversible terminator approach. Typically, the Illumina Genome Analyzer System utilizes flow-cells with 8 channels, generating sequencing reads of 18 to 36 bases in length, generating >1.3 Gbp of high quality data per run (see www.illumina.com).

In yet a further sequence by synthesis process, the incorporation of differently labeled nucleotides is observed in real time as template dependent synthesis is carried out. In particular, an individual immobilized primer/template/polymerase complex is observed as fluorescently labeled nucleotides are incorporated, permitting real time identification of each added base as it is added. In this process, label groups are attached to a portion of the nucleotide that is cleaved during incorporation. For example, by attaching the label group to a portion of the phosphate chain removed during incorporation, i.e., a β, γ, or other terminal phosphate group on a nucleoside polyphosphate, the label is not incorporated into the nascent strand, and instead, natural DNA is produced. Observation of individual molecules typically involves the optical confinement of the complex within a very small illumination volume. By optically confining the complex, one creates a monitored region in which randomly diffusing nucleotides are present for a very short period of time, while incorporated nucleotides are retained within the observation volume for longer as they are being incorporated. This results in a characteristic signal associated with the incorporation event, which is also characterized by a signal profile that is characteristic of the base being added. In related aspects, interacting label components, such as fluorescent resonant energy transfer (FRET) dye pairs, are provided upon the polymerase or other portion of the complex and the incorporating nucleotide, such that the incorporation event puts the labeling components in interactive proximity, and a characteristic signal results, that is again, also characteristic of the base being incorporated (See, e.g., U.S. Pat. Nos. 6,056,661, 6,917,726, 7,033,764, 7,052,847, 7,056,676, 7,170,050, 7,361,466, 7,416,844 and Published U.S. Patent Application No. 2007-0134128, the full disclosures of which are hereby incorporated herein by reference in their entirety for all purposes).

In some embodiments, the nucleic acids in the sample can be sequenced by ligation. This method uses a DNA ligase enzyme to identify the target sequence, for example, as used in the polony method and in the SOLiD technology (Applied Biosystems, now Invitrogen). In general, a pool of all possible oligonucleotides of a fixed length is provided, labeled according to the sequenced position. Oligonucleotides are annealed and ligated; the preferential ligation by DNA ligase for matching sequences results in a signal corresponding to the complementary sequence at that position.

### Kits

Any of the compositions described herein may be comprised in a kit. In a non-limiting example the kit, in suitable container means, comprises: one or more primers, a reverse transcription enzyme and optionally reagents for amplification.

The containers of the kits will generally include at least one vial, test tube, flask, bottle, syringe or other containers, into which a component may be placed, and preferably, suitably aliquotted. Where there is more than one component in the kit, the kit also will generally contain a second, third or other additional container into which the additional components may be separately placed. However, various combinations of components may be comprised in a container.

When the components of the kit are provided in one or more liquid solutions, the liquid solution can be an aqueous solution. However, the components of the kit may be provided as dried powder(s). When reagents and/or components are provided as a dry powder, the powder can be reconstituted by the addition of a suitable solvent.

A kit will preferably include instructions for employing, the kit components as well the use of any other reagent not included in the kit. Instructions may include variations that can be implemented.

### EXAMPLES

### Example 1

Reaction Conditions and Reagents:

In this example cDNA was generated from total RNA (10 ng total Brain RNA, Ambion). First strand cDNA was generated using a variety of tailed first primers, which differ in the 5′-tail sequence. Two different reverse transcriptases (RTa and RTb) were used with the various first strand primers. The reaction conditions, both buffer compositions and incubation conditions, were selected for the given RT according to the manufacturer's suggestions. For RTa in this and the following example, first and second cDNA reagents and reaction conditions were those of Ovation Pico (NuGEN Technologies). The RTb reagents and reaction conditions were those of Ovation One Direct RNA amplification (NuGEN Technologies). The various first strand primers employed in the example comprise a 3′-end random hexamer sequence. First strand primers A, B, and C were tested, as well as a mixture of primer A comprising random hexamer at the 3′-end and a primer comprising a poly dT sequence at the 3′-end and the same 5-RNA tail as the A first strand primers. Primers A, B, and C are chimeric primers comprising a RNA tail. cDNA was also generated with tailed all DNA first primers which are the same sequence composition as the A or B or C. cDNA was also generated using N6 or N9 primers, representing a hexamer or a nine-mer of a random DNA sequence. The cDNA generated using these non tailed primers equally represented the total RNA as generated by the corresponding RT. These non-tailed random primers are commonly used for random priming to generate cDNA from RNA.

Second strand synthesis was carried out as per the manufacturer's instruction (Ribo-SPIA second strand synthesis).

Further amplification of the cDNA was carried out following generation of the double stranded cDNA by SPIA, employing chimeric amplification primers comprising a DNA sequence at the 3′-end and an RNA sequence at the 5′-end, which are the same sequence as the corresponding tail of the first strand primers. SPIA amplification reaction conditions and components were the same for all reactions, regardless of RT selection. SPIA amplification was carried out according to manufacturer instructions, with chimeric amplification primer corresponding to the first primer(s) utilized in the first strand synthesis.

Non-amplified (double stranded cDNA) and amplified cDNA quantification was carried out by real-time qPCR with SYBR green, using specific primer pairs for the various RNA components (18S rRNA, 28S rRNA, various mRNA, 12S mitochondrial RNA (mtrRNA), 16S mtrRNA, and various mitochondrial transcripts) as shown. PCR primers were designed to interrogate/quantify different sequence regions along the test transcripts. Real-time PCR was carried out following the dilution of aliquots taken at the end of second strand synthesis (non-amplified cDNA) or at the end of linear amplification (amplified cDNA). The aliquots were diluted in Tris-EDTA and 2u1 were added to the real time PCR reactions.

Results:

Real time PCR quantification of a) non-amplified cDNA of rRNA (18S and 28S), b) mRNA (specific mRNAs as stated), c) mitochondrial rRNA (mt rRNA, 12S and 16S) and d) mitochondrial gene transcripts demonstrated reduced reverse transcription of rRNA as compared to other transcripts. This reduced reverse transcription of rRNA is affected in part by the choice of RT and in part by the choice of the sequence of tailed first strand random primers, as shown in the figures.

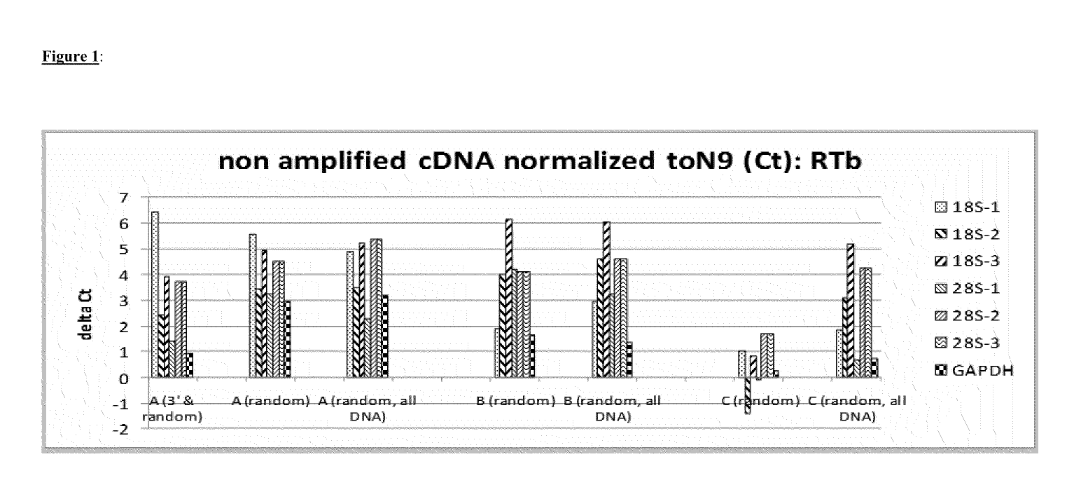

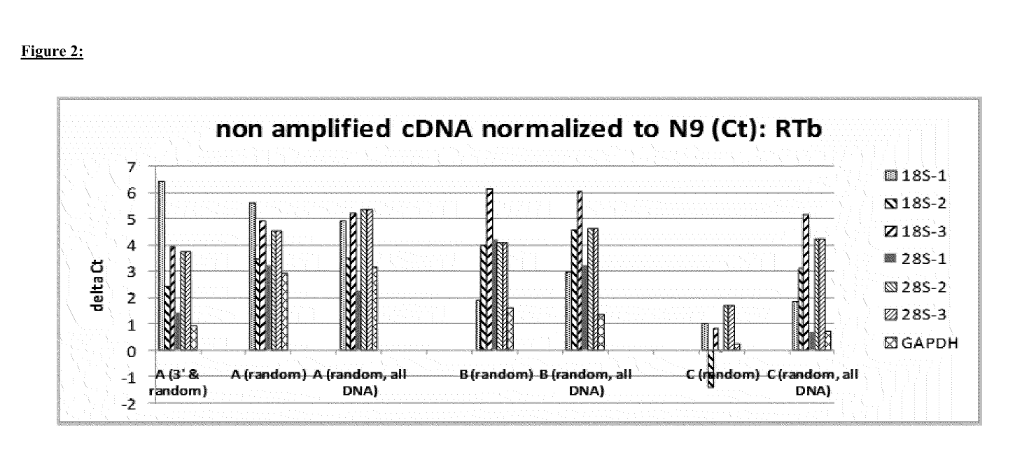

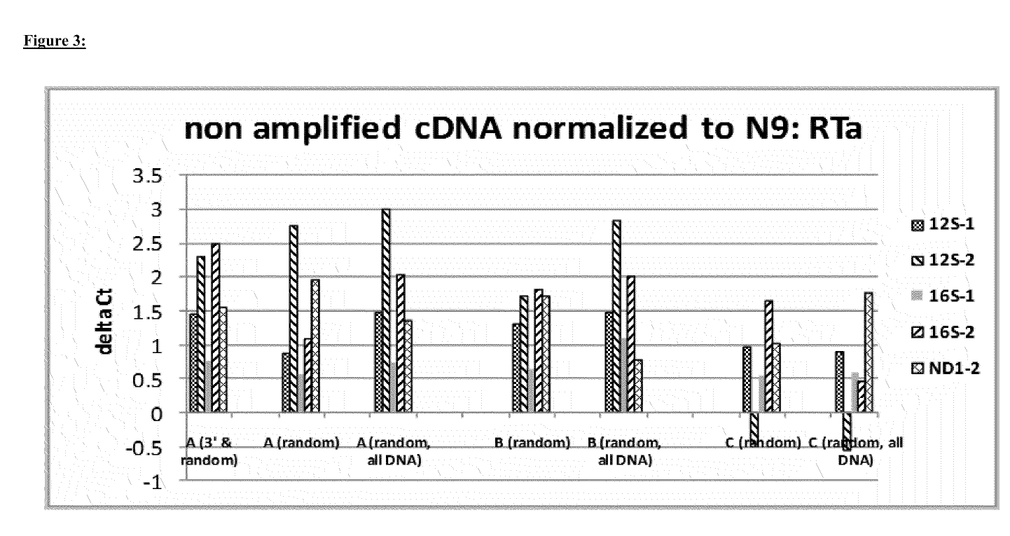

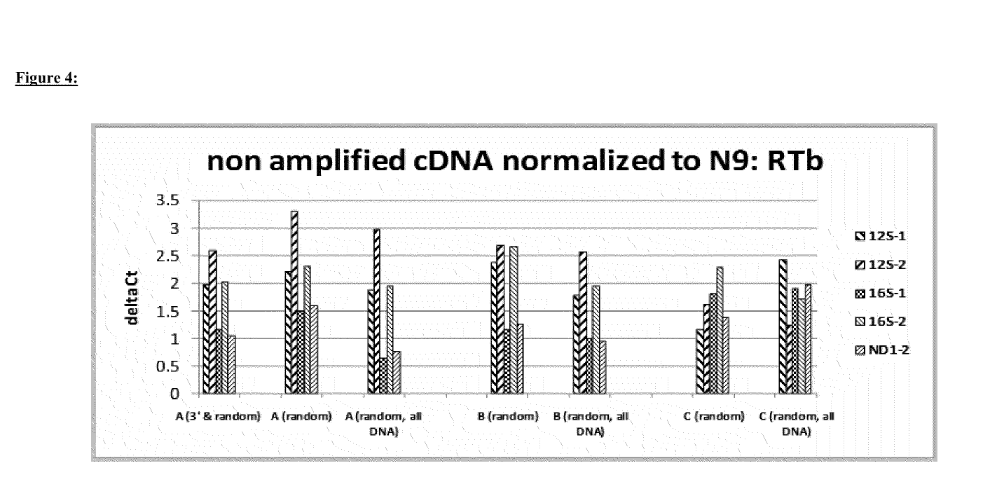

FIGS. 1-4: The effect of the choice of tailed first primer on the efficiency of reverse transcription of rRNA was reduced efficiency of reverse transcription (higher delta Ct of cDNA normalized to N9), dependent on the choice of the first primer.

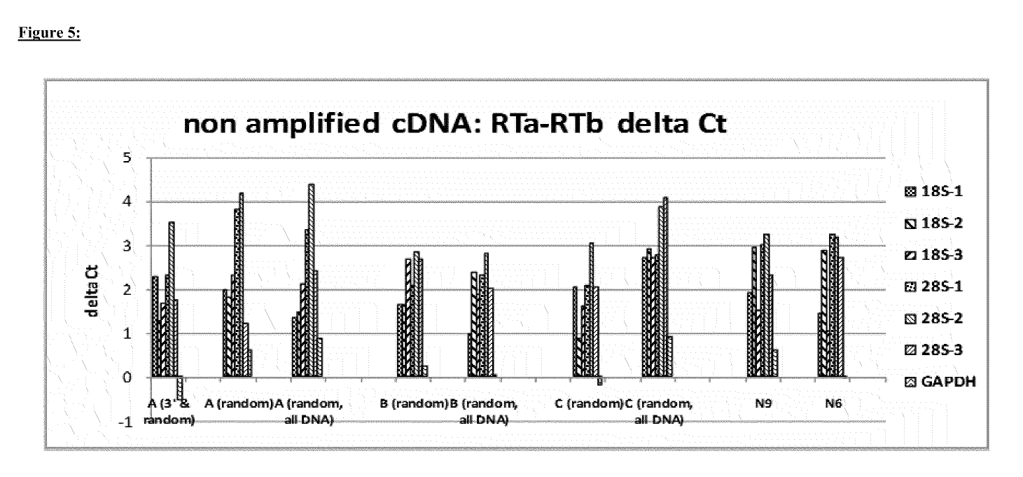

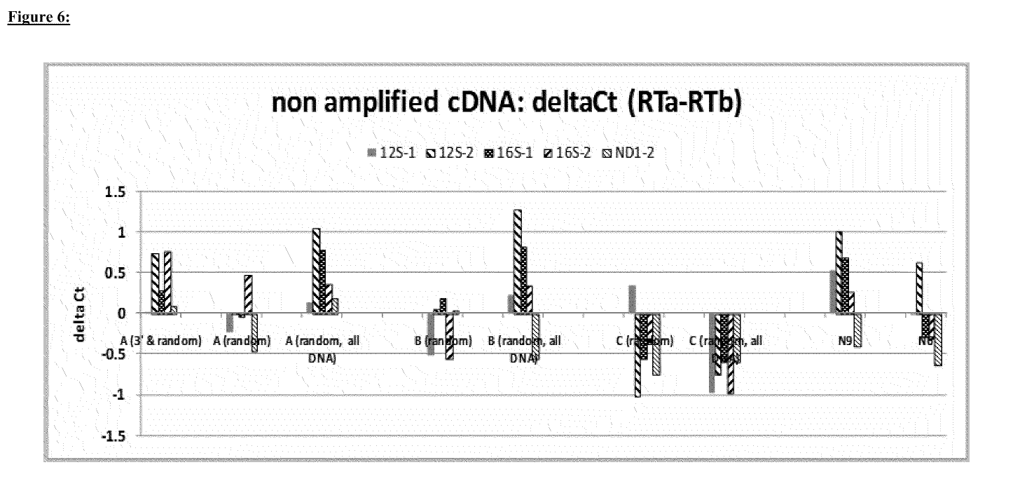

FIGS. 5-6: The efficiency of reverse transcription of rRNA was affected by the choice of RT. The effect of the choice of RT on the efficiency of transcription was determined by the delta Ct for the various non-amplified cDNA (real time PCR) generated with the same first primers and different RTs (RTa or RTb). Here, delta Ct equals Ct for RTa minus Ct for RTb

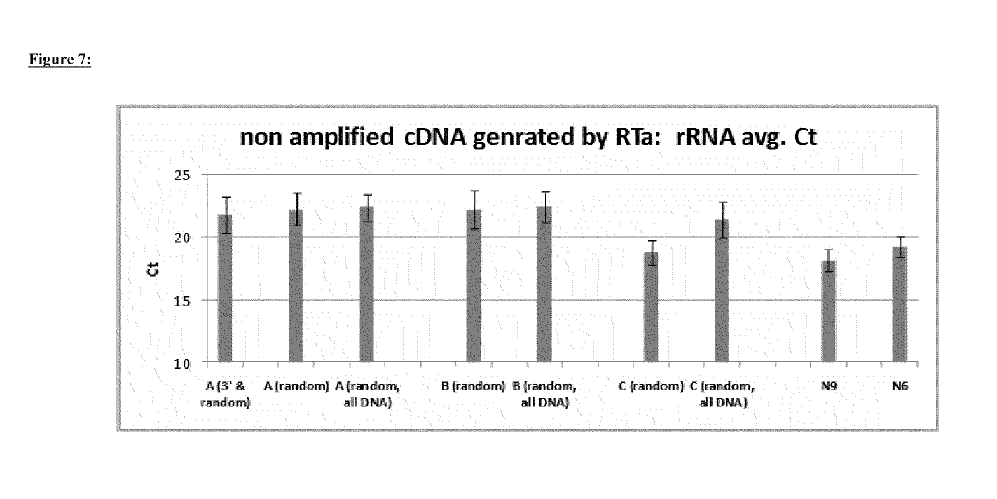

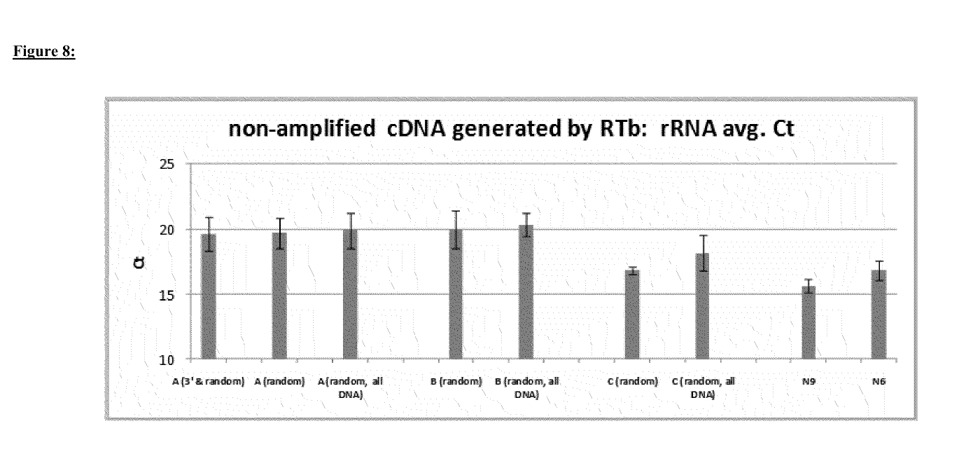

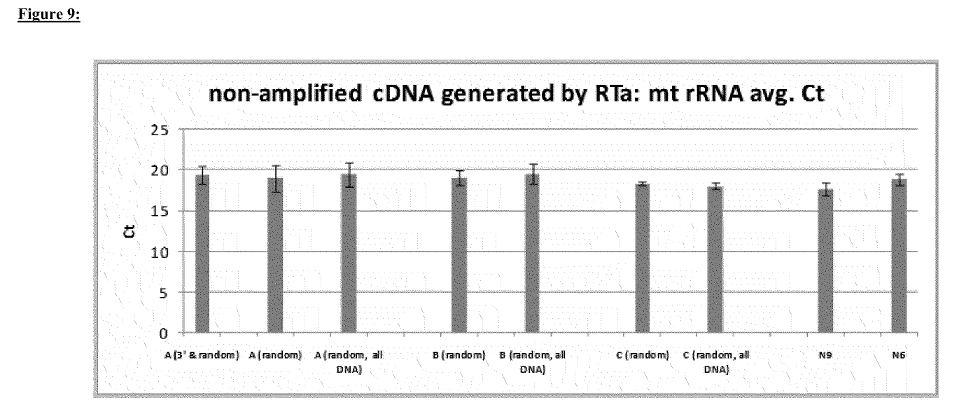

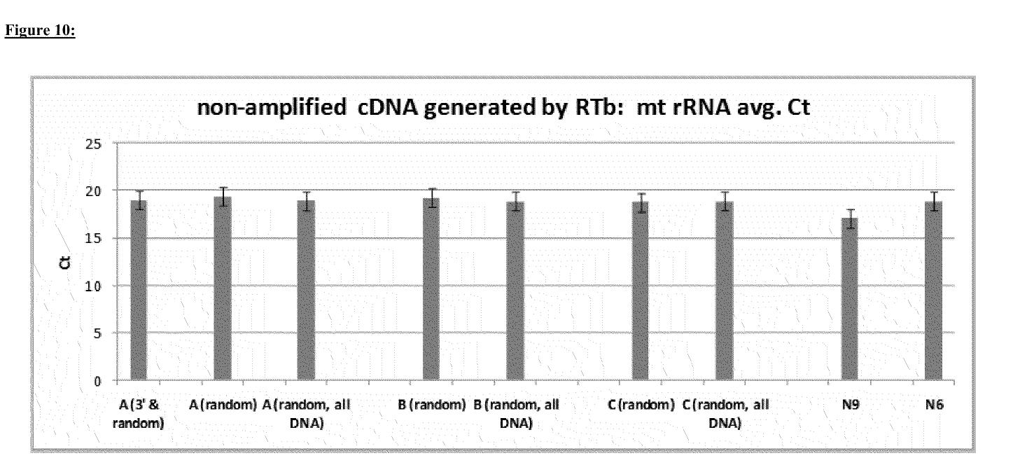

FIGS. 7-10: Another result of the effect of the choice of the tailed first strand cDNA primer and the RT employed for the generation of the non-amplified cDNA, is shown. Specifically, shown is the quantification of the various regions on rRNA as was carried out by real time PCR. The average Ct value across all the regions for a given first strand tailed primer and RT (avg. Ct), and standard deviation across these values as well as the non tailed random primers, N9 and N6, is shown. The rRNA content is affected by choice of first strand primer, and to a lesser extent by the RT. The effect on mitochondrial rRNA is minimal by both. The overall effect on the content of rRNA in the non amplified cDNA, and subsequently in the amplified cDNA, is greater than a 10-fold reduction (delta Ct greater than 3), thus rendering the methods of the inventions most effective relative to other methods such as rRNA removal by enzymatic digestions, hybridization, or selective transcription of only mRNAs with polyA sequences.

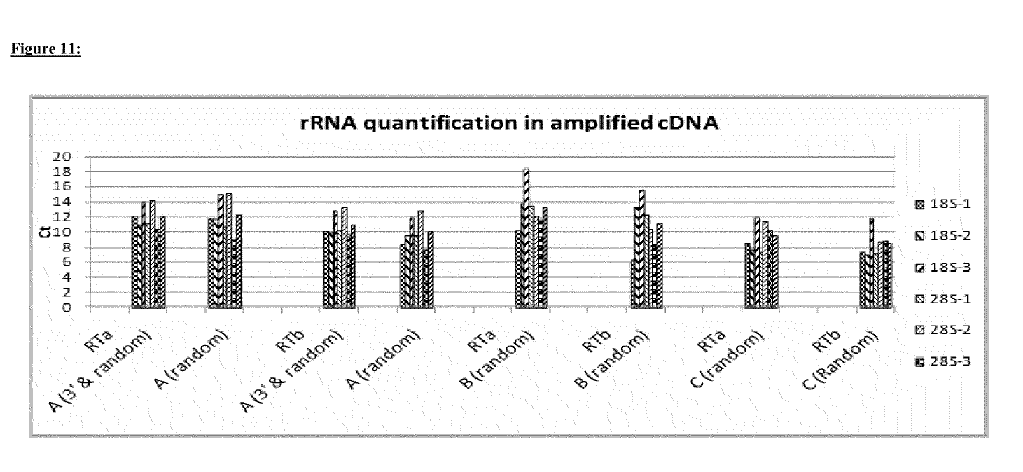

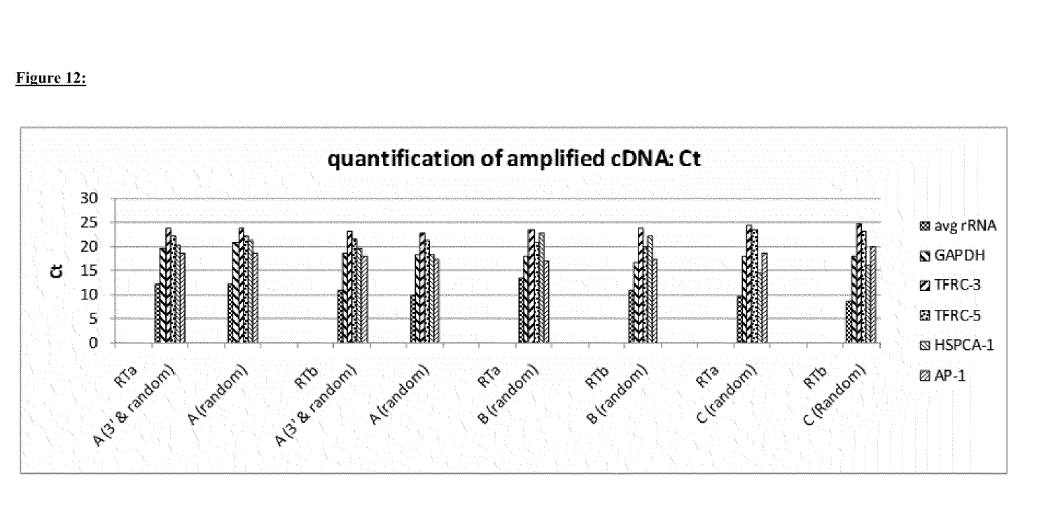

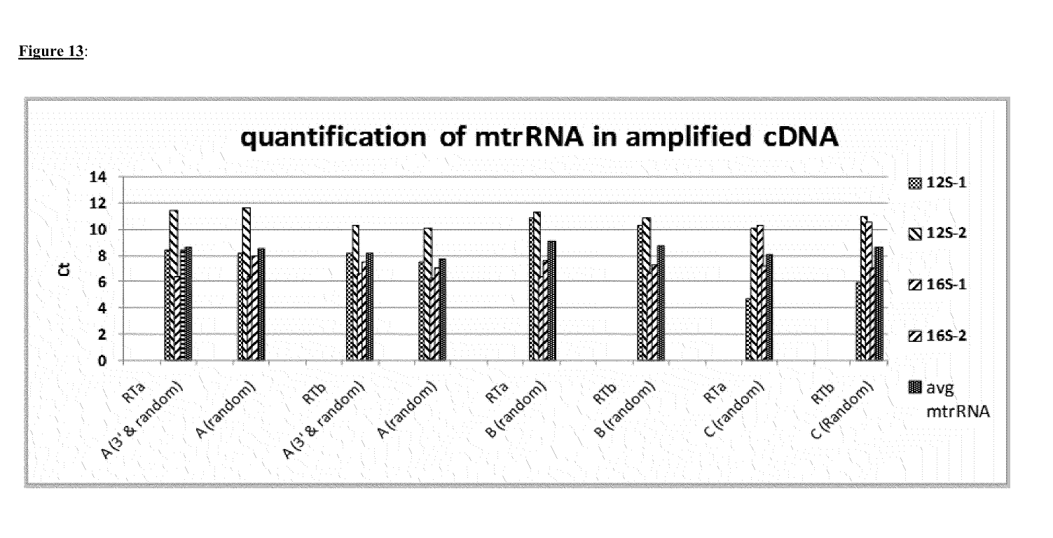

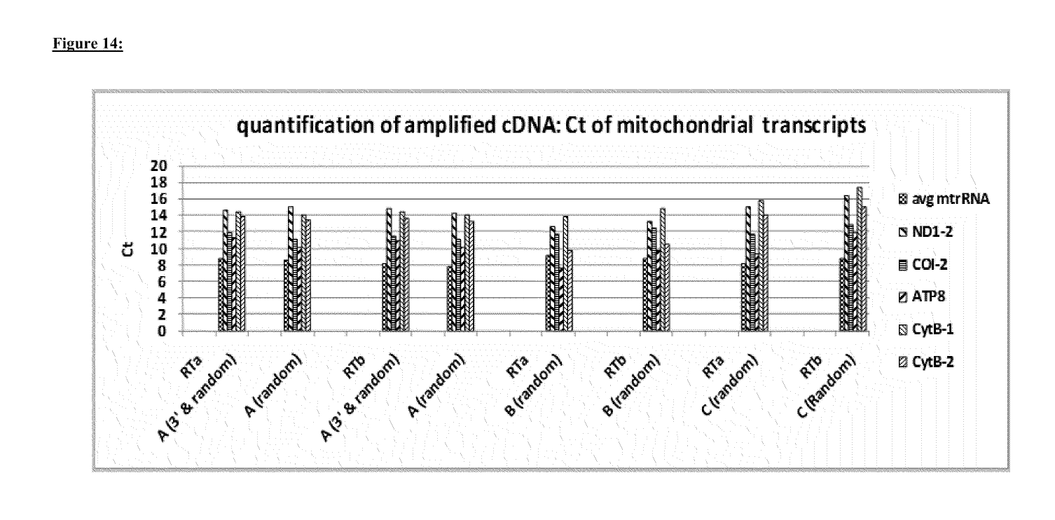

FIGS. 11-14: Real time PCR quantification of amplified cDNA of rRNA (18S and 28S), mRNA (specific mRNAs as stated), mitochondrial rRNA (mt rRNA, 12S and 16S) and mitochondrial gene transcripts, was carried out following single primer linear isothermal amplification (SPIA). The results, shown in the figures, demonstrate that the pattern of relative reduction of the content of rRNA shown above in the corresponding non-amplified cDNA is maintained in the amplified cDNA. The results further demonstrate that, as shown for non-amplified cDNA, there is a minimal effect of primer selection or RT on the content of mrtRNA in the amplified cDNA generated from total RNA.

### Example 2

This example describes reduction in the representation of rRNA in amplified cDNA from C. elegans total RNA. Non-amplified cDNA was prepared priming with Primer A (3′ & random) or Primer B (3′ & random) and RTa or RTb and using first and second strand reagents and protocol. Total C. elegans RNA input was 2 ng. The cDNA was further amplified with two different reagent and protocol systems: SPIA amplification using reagents and protocols from the Ovation Pico kit (NUGEN) or reagents and protocol from WGA-Ovation kit (NuGEN). rRNA and non rRNA cDNA in the double stranded cDNA generated by the different primer sets, RT and protocols were quantified by qPCR as described in Example 1.

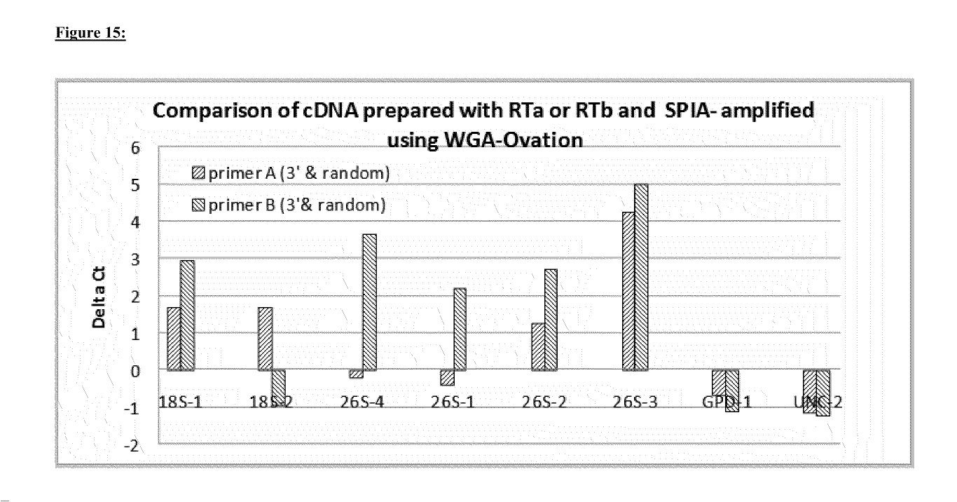

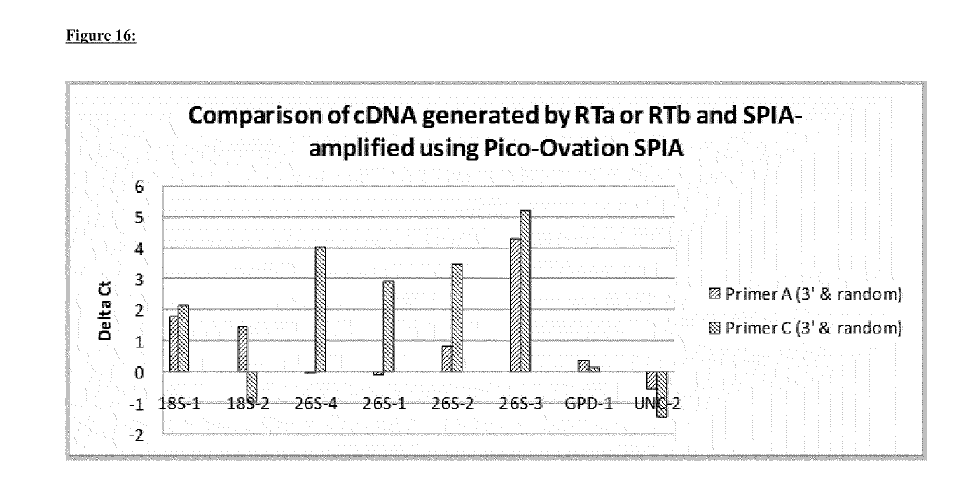

FIGS. 15 and 16 represent the delta CT (dCT) of quantification of the same transcript loci in amplified cDNA prepared with different first strand RT and amplified with the same SPIA amplification reagents and protocols. Similar differential reduction of rRNA, but not non rRNA or mRNA, is observed in products generated by the same SPIA amplification reagents and protocol.

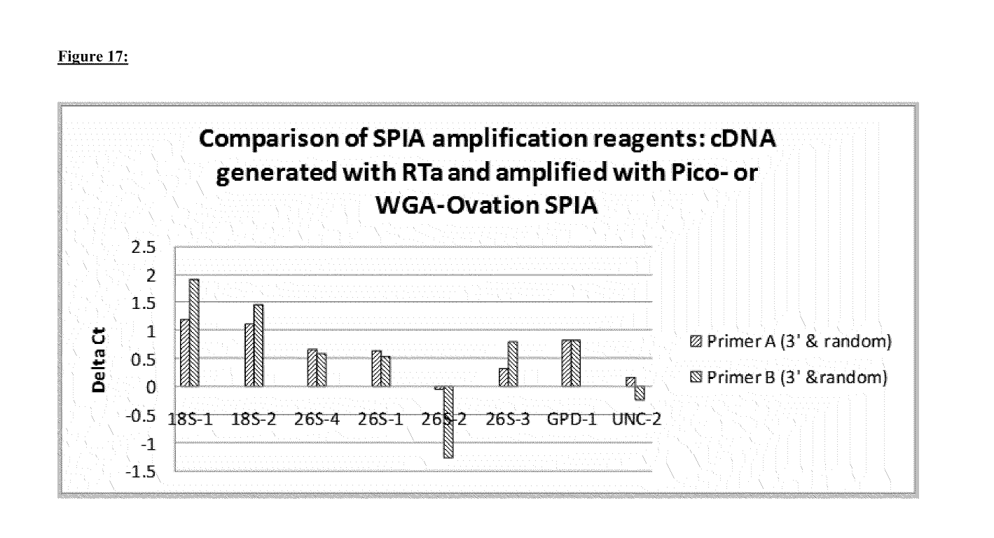

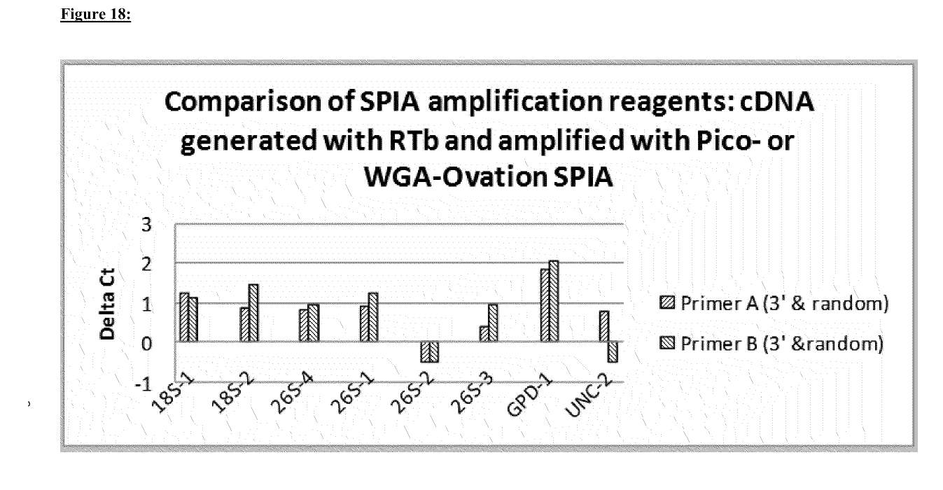

FIGS. 17 and 18 illustrate quantification of rRNA and mRNA in amplified cDNA prepared by cDNA synthesis using the same RT and cDNA reagents and protocol (RTa in FIG. 17 and RTb in FIG. 18) and amplified by different SPIA reagents (SPIA amplification from Ovation Pico kit and SPIA amplification from WGA-Ovation kit). rRNA and mRNA representation is not differentially affected by the SPIA amplification system.

The results in this example indicate that the reduced representation of rRNA in the amplified cDNA is dependent on the combination of primer set and cDNA synthesis (RTa and RTb).

### Example 3

This example describes differential reduction in rRNA representation in amplified cDNA from C. elegans total RNA as measured by mass normalized quantification by qPCR and massively parallel sequencing (RNA-Seq) using Illumina's Genome Analyzer.

Quantification of rRNA and mRNA in purified amplification products generated by RTa (Ovation Pico cDNA) with two primer sets demonstrate the reduction of rRNA representation in the products generated with one of the two primer sets relative to the other. The effect is independent of the SPIA amplification reagent and conditions, as demonstrated by results from products generated using the reagents and conditions for SPIA amplification reaction from the Ovation Pico or WG-Ovation kits.

Quantification by qPCR of rRNA was carried out with 10 pg input (amplified products) and that of the mRNA transcripts with 10 ng input (amplified products). The corrected dCt reflects the correction of the rRNA values to 10 ng input (mass normalized).

The two data sets represented in Table 1 are from two separate experiments each carried out in duplicate, and demonstrate the reproducibility of the amplification reactions. The corrected dCt equals the average Ct for mRNA minus the average Ct for rRNA, for products generated using each of the defined conditions. The delta Ct for products generated by the Primer C set and RTa (cDNA synthesis with Ovation Pico reagents and reaction conditions) in both experiments is about 4 Ct lower than that obtained with the primer A set. This difference, about 10 fold, is reflected in the representation of the rRNA detected by sequencing, Table 3. The data obtained from transcripts quantified by qPCR provides a rough estimate of the representation of the rRNA in the total product of the amplified cDNA.

A more accurate value of the rRNA representation in the amplified cDNA can be achieved from massively parallel sequencing enabled by the Next Generation Sequencing technologies and platforms, as represented by the RNA-Seq data using Illumina's Genome Analyzer. Sequencing data were obtained for two C. elegans libraries generated from amplified cDNA prepared by the primer sets and protocols described above. For sequencing ready library preparations, purified amplified cDNA was made double stranded by random priming (NuGEN Exon kit). Library preparation for sequence analysis on Illumina Genome Analyzer was carried out using Illumina's protocols and reagents. The sequence data was generated from a single lane run according to the manufacturer instructions. The two libraries were prepared at different times and by different laboratories. In so far as the rRNA sequencing reads are the highest abundant species the sequencing data for both libraries are suitable for the estimation of the effect of rRNA representation in the amplified cDNA. The reduced representation of rRNA in the total amplified cDNA prepared with primer C set, RTa cDNA synthesis, and WGA-Ovation SPIA amplification, as compared to that prepared with primer set A, RTa cDNA synthesis and Ovation-Pico SPIA amplification, is shown in table 2. Specifically, Table 2 provides a summary of C. elegans rRNA representation in RNA-Seq data from Illumina Genome Analyzer IIx sequencing data generated with two libraries. Column A provides results for a library prepared from amplified cDNA generated with primer A (3′ & random), RTa cDNA (Ovation Pico), and SPIA amplification using the Ovation Pico reagents. Column C provides results for a library prepared from amplified cDNA generated with primer C (3′ & random), RTa cDNA (Ovation Pico), and SPIA amplification using the WG-Ovation reagents.

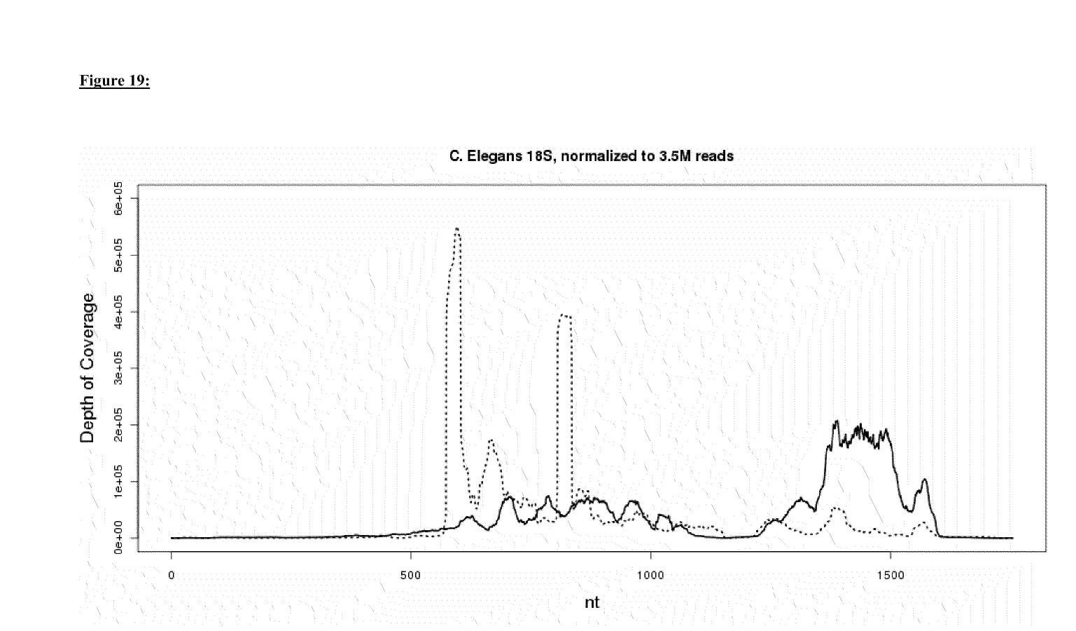

Further indication of the dependence of the effect of the efficiency of first strand cDNA priming and reverse transcription on the rRNA representation is demonstrated from the analysis of alignment of the rRNA sequencing reads to the rRNA sequences, as shown in FIGS. 18 and 19. The solid line corresponds to the depth of coverage that is derived from the library prepared from amplified cDNA using Primer A (3′ & random), RTa and SPIA amplification using the reagents and protocols from Ovation Pico kit, and the dashed line corresponds to the depth of coverage that is derived from the library prepared from amplified cDNA using primer C (3′ & random), RTa and SPIA amplification using the reagents and protocols from WGA-Ovation kit.

TABLE 1

| | Avg. | Avg. non | | |
|---|---|---|---|---|
| | rRNA Ct | rRNA Ct | dCt | |
| | (10 pg) | (10 ng) | corrected | ddCt |
| Primer A (3′ & random) | 13.4 | 21.5 | 14.1 | |
| Pico SPIA 1 | | | | |
| Primer A (3′ & random) | 13.3 | 21.4 | 14.1 | |
| WGA SPIA 2 | | | | |
| Primer C (3′ & random) | 16.7 | 21.3 | 10.5 | 3.6 |
| Pico SPIA 1 | | | | |
| Primer C (3′ & random) | 16.1 | 20.0 | 9.9 | 4.1 |
| WGA SPIA 2 | | | | |

TABLE 2

| | A | C |
|---|---|---|
| Reads | 15722172 | 10922087 |
| rRNA | 10008595 | 3646359 |
| ws190 aligned reads | 10319515 | 4343998 |
| % non-ribosomal WS190 | 3.00% | 16.10% |
| aligned reads | | |
| % rRNA | 97.00% | 83.90% |
| 18S | 2123935 | 2028797 |
| 23S | 1376060 | 1471198 |

TABLE 3
Expected differential reduction of % rRNA representation
relative to non-ribosomal transcripts.

| | | | Calculated rRNA | | |
|---|---|---|---|---|---|
| | Non ribosomal | | % of | Fold | delta Ct |
| | RNA | rRNA | total | reduction | (relative to A) |
| A | 3 | 97 | 97% | | |
| B | 3 | 9.7 | 76% | 10 | 3.3 |
| C | 3 | 3 | 50% | 32.3 | 5.0 |
| D | 3 | 0.3 | 9% | 323.3 | 8.3 |

### Example 4

This example describes reduction in rRNA representation in amplified cDNA from different human total RNA samples, measured by massively parallel sequencing using Illumina Genome Analyzer IIx (RNA-Seq).

Reduced representation of rRNA in amplified cDNA is demonstrated by RNA-Seq data obtained for different amplified cDNA prepared from different human total RNA samples. The samples analyzed in this example represent both high quality total RNA (human brain reference RNA) and low quality total RNA derived from FFPE sample (colon tumor). The details of the samples used, input into the amplification using various NuGEN's RNA amplification kits, reagents and protocols for first strand cDNA synthesis and SPIA amplification, and library preparation (Encore; NuGEN Technologies) for the massively parallel sequencing are summarized in Table 4.

The rRNA representation in the total amplified cDNA obtained from the sequencing data for the various preparations and samples is summarized in Table 5. In Table 5, 103803-1 and -2 are replicate amplifications from same RNA; and 6957-1 and -2 are replicate libraries prepared from a single amplification. rRNA representation is shown to be affected by the overall reagents and protocols used for the preparation of amplified cDNA, and marked reduction in rRNA representation is shown for all samples, including those derived from poor quality RNA derived from FFPE samples.

TABLE 4

| | | | | 1st | | |
|---|---|---|---|---|---|---|
| | | | 1st strand | strand | | |
| | | | cDNA | cDNA | SPIA | Library |
| Sample | RNA | Input | primer | protocol | protocol | prep |
| FFPE | FFPE colon | 100 ng | RNA-SEQ | FFPE | FFPE | Encore |
| | tumor | | | | | |
| RNA- | FFPE colon | 100 ng | RNA-SEQ | FFPE | Pico | Encore |
| SEQₘ | tumor | | | | | |
| RNA- | Human brain | 10 ng | RNA-SEQ | Pico | Pico | Encore |
| SEQ | reference | | | | | |

TABLE 5

| | | rRNA |
|---|---|---|
| FFPE | 103803-1 | 31.4% |
| | 103803-2 | 27.6% |
| | 6957-1 | 30.6% |
| | 6957-2 | 29.0% |
| RNA-SEQₘ | 103803-1 | 13.8% |
| | 103803-2 | 14.9% |
| | 6957-1 | 15.2% |
| | 6957-2 | 15.9% |
| RNA-SEQ | Human brain | 5.8% |
| | RNA reference | |

Table 6 provides sequences of example primers for reverse transcription (“random”), cDNA second strand synthesis (“3′”), and SPIA primers useful in the methods and compositions of the invention. Ribonucleotides appear in lower case and deoxyribonucleotides appear in upper case. “N” represents a random nucleotide.

TABLE 6

| Primer ID | Sequence | Length |
|---|---|---|
| A SPIA | cuucuauaguuuagguaacuuug | 30 |
| | TGTTTGA | |
| A random | cuucuauaguuuagguaacuuuguguuuga | |
| | NNNNNN | |
| A 3′ | cuucuauaguuuagguaacuuuguguuuga | |
| | TTTTTTTTTTTTTNN | |
| B SPIA | gguaauacgacucacuau | 25 |
| | AGGCAGA | |
| B random | gguaauacgacucacuauaggcaga | |
| | NNNNNN | |
| B 3′ | gguaauacgacucacuauaggcaga | |
| | TTTTTTTTTTTTTNN | |
| C SPIA | gacggaugcggucu | 21 |
| | CCAGTGT | |
| C random | gacggaugcggucuccagugu | |
| | NNNNNN | |
| C 3′ | gacggaugcggucuccagugu | |
| | TTTTTTTTTTTTTNN | |
| D SPIA | cccuccaaggctccc | 22 |
| | CAGTATC | |
| D random | cccuccaaggctccccaguauc | |
| | NNNNNN | |
| D 3′ | cccuccaaggctccccaguauc | |
| | TTTTTTTTTTTTTNN | |
| E SPIA | agggugagaaaggcc | 22 |
| | GGAGACA | |
| E random | agggugagaaaggccggagaca | |
| | NNNNNN | |
| E 3′ | agggugagaaaggccggagaca | |
| | TTTTTTTTTTTTTNN | |
| F SPIA | acacauacgauuuagguga | 25 |
| | CACTAT | |
| F random | acacauacgauuuaggugacacuau | |
| | NNNNNN | |
| F 3′ | acacauacgauuuaggugacacuau | |
| | TTTTTTTTTTTTTNN | |
| G SPIA | cguuaaguaugaacccag | 25 |
| | ATGTATT | |
| G random | cguuaaguaugaacccagauguauu | |
| | NNNNNN | |
| G 3′ | cguuaaguaugaacccagauguauu | |
| | TTTTTTTTTTTTTNN | |
| H SPIA | gaagacagauggugc | 22 |
| | AGCCACA | |
| H random | gaagacagauggugcagccaca | |
| | NNNNNN | |
| H 3′ | gaagacagauggugcagccaca | |
| | TTTTTTTTTTTTTNN | |
| I SPIA | cguauucugacgacguacuc | 27 |
| | TCAGCCT | |
| I random | cguauucugacgacguacucucagccu | |
| | NNNNNN | |
| I 3′ | cguauucugacgacguacucucagccu | |
| | TTTTTTTTTTTTTNN | |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for differentially reducing the reverse transcription of ribosomal RNA (rRNA) from an RNA sample comprising:
(a) providing one or more primers of known sequence, each of the one or more primers comprising a 3′ portion hybridizable to one or more target RNAs in the RNA sample, wherein said hybridizable 3′ portion comprises a randomly generated sequence;
(b) combining the one or more primers with a reverse transcriptase (RT); and
(c) reverse transcribing the RNA sample, thereby producing reverse transcribed products, wherein the reverse transcription of rRNA is reduced.

2. (canceled)

3. The method of claim 1, further comprising step (d) amplifying said reverse transcribed products, thereby producing amplified products.

4. The method of claim 1, further comprising sequencing said reverse transcribed products.

5. The method of claim 4, wherein said reverse transcribed products are amplified prior to sequencing.

6. The method of claim 1, wherein the one or more primers are tailed primers comprising a 3′ portion hybridizable to one or more target RNAs in the RNA sample and a 5′ portion that is not hybridizable to the one or more target RNAs in the RNA sample.

7. The method of claim 1, wherein the one or more primers are chimeric primers comprising a DNA portion and an RNA portion.

8. The method of claim 7, wherein the chimeric primers comprise a 3′-DNA portion hybridizable to one or more target RNAs in the RNA sample and a 5′-RNA portion that is not hybridizable to the one or more target RNAs in the RNA sample.

9. (canceled)

10. The method of claim 6, wherein the hybridizable 3′ portion of the primers comprise a length of nucleotides selected from the group consisting of 5-15 nucleotides and 6 nucleotides.

11. (canceled)

12. (canceled)

13. The method of claim 6, wherein the primer is one of a plurality of primers of known sequence, each primer comprising a 3′ portion hybridizable to the sample RNA and a 5′ portion that is not hybridizable to the sample RNA, wherein each of the non-hybridizable 5′ portions of the primers comprise the same sequence.

14. (canceled)

15. The method of claim 1, wherein the RNA is selected from the group consisting of total RNA, mitochondrial RNA, chloroplast RNA, DNA-RNA hybrids, viral RNA, cell free RNA, and mixtures thereof.

16. (canceled)

17. The method of claim 1, wherein the RT is selected from the group consisting of: a Moloney murine leukemia virus RT, a human immunodeficiency virus RT, a rous sarcoma virus RT, an avian myeloblastosis virus RT, a rous associated virus RT, a myeloblastosis associated virus RT, an avian sarcoma-leukosis virus RT, an RT lacking RNase H activity, modified RTs derived therefrom, and combinations thereof.

18. A method of identifying a primer sequence that differentially reduces the reverse transcription of ribosomal RNA (rRNA) from an RNA sample comprising:
(a) providing one or more primers of known sequence;
(b) combining the one or more primers with a reverse transcriptase (RT) to reverse transcribe the RNA sample, thereby producing reverse transcribed products;
(c) optionally generating double-stranded cDNA products from said reverse transcribed products;
(d) optionally amplifying said double-stranded cDNA products; and
(e) analyzing the reverse transcribed products to determine if the reverse transcription of rRNA is reduced by the primer sequence.

19. The method of claim 1, wherein the one or more primers are tailed primers comprising a 3′ portion hybridizable to one or more target RNAs in the RNA sample and a 5′ portion that is not hybridizable to the one or more target RNAs in the RNA sample.

20. The method of claim 18, wherein the one or more primers are chimeric primers comprising a DNA portion and an RNA portion.

21. (canceled)

22. (canceled)

23. The method of claim 20, wherein the chimeric primers comprise a 3′-DNA portion hybridizable to one or more target RNAs in the RNA sample and a 5′-RNA portion that is not hybridizable to the one or more target RNAs in the RNA sample.

24. The method of claim 19, wherein the hybridizable 3′ portion of the primers comprise a randomly generated sequence.

25. The method of claim 19, wherein the hybridizable 3′ portion of the primers comprise a length of nucleotides selected from the group consisting of 5-15 nucleotides and 6 nucleotides.

26. (canceled)

27. (canceled)

28. The method of claim 19, wherein the primer is one of a plurality of primers of known sequence, each primer comprising a 3′ portion hybridizable to the sample RNA and a 5′ portion that is not hybridizable to the sample RNA, wherein each of the non-hybridizable 5′ portions of the primers comprise the same sequence.

29. The method of claim 19, wherein the primer sequence that reduces the reverse transcription of ribosomal RNA is identified based on varying the non-hybridizable 5′ portion of the one or more primers.

30. The method of claim 19, wherein the primer sequence that reduces the reverse transcription of ribosomal RNA is identified based on varying the RT used in step (e).

31. (canceled)

32. The method of claim 18, wherein the RNA is selected from the group consisting of total RNA, mitochondrial RNA, chloroplast RNA, DNA-RNA hybrids, viral RNA, cell free RNA, and mixtures thereof.

33. The method of claim 18, wherein the RT is selected from the group consisting of: a Moloney murine leukemia virus RT, a human immunodeficiency virus RT, a rous sarcoma virus RT, an avian myeloblastosis virus RT, a rous associated virus RT, a myeloblastosis associated virus RT, an avian sarcoma-leukosis virus RT, an RT lacking RNase H activity, modified RTs derived therefrom, and combinations thereof.

34. A method of identifying a reverse transcriptase enzyme (RT) that differentially reduces the reverse transcription of rRNA from an RNA sample comprising:
(a) providing one or more primers of known sequence;
(b) combining the one or more primers with an RT to reverse transcribe the RNA sample, thereby producing reverse transcribed products;
(c) optionally generating double-stranded cDNA products from said reverse transcribed products;
(d) optionally amplifying said double-stranded cDNA products; and
(e) analyzing the reverse transcribed products to determine if the reverse transcription of rRNA is reduced by the RT.

35. The method of claim 34, wherein the one or more primers are tailed primers comprising a 3′ portion hybridizable to one or more target RNAs in the RNA sample and a 5′ portion that is not hybridizable to the one or more target RNAs in the RNA sample.

36. The method of claim 34, wherein the one or more primers are chimeric primers comprising a DNA portion and an RNA portion.

37. (canceled)

38. (canceled)

39. The method of claim 36, wherein the chimeric primers comprise a 3′-DNA portion hybridizable to one or more target RNAs in the RNA sample and a 5′-RNA portion that is not hybridizable to the one or more target RNAs in the RNA sample.

40. The method of claim 35, wherein the hybridizable 3′ portion of the primers comprise a randomly generated sequence.

41. The method of claim 35, wherein the hybridizable 3′ portion of the primers comprise a length of nucleotides selected from the group consisting of 5-15 nucleotides and 6 nucleotides.

42. (canceled)

43. (canceled)

44. The method of claim 35, wherein the primer is one of a plurality of primers of known sequence, each primer comprising a 3′ portion hybridizable to the sample RNA and a 5′ portion that is not hybridizable to the sample RNA, wherein each of the non-hybridizable 5′ portions of the primers comprise the same sequence.

45. The method of claim 35, wherein the primer sequence that reduces the reverse transcription of ribosomal RNA is identified based on varying the non-hybridizable 5′ portion of the one or more primers.

46. The method of claim 35, wherein the primer sequence that reduces the reverse transcription of ribosomal RNA is identified based on varying the RT used in step (e).

47. (canceled)

48. The method of claim 34, wherein the RNA is selected form the group consisting of total RNA, mitochondrial RNA, chloroplast RNA, DNA-RNA hybrids, viral RNA, cell free RNA, and mixtures thereof.

49. The method of claim 34, wherein the RT is selected from the group consisting of: a Moloney murine leukemia virus RT, a human immunodeficiency virus RT, a rous sarcoma virus RT, an avian myeloblastosis virus RT, a rous associated virus RT, a myeloblastosis associated virus RT, an avian sarcoma-leukosis virus RT, an RT lacking RNase H activity, modified RTs derived therefrom, and combinations thereof.
